(19) United States
(12) Patent Application Publication
(10) Pub. No.: US 2006/0067905 A1
(43) Pub. Date: Mar. 30, 2006

(54) Formulations and method for treating baldness

(75) Inventors: Karl Lintner, Rambouillet (FR); Claire Maschamberlin, Chevreuse (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: SEDERMA SAS, Le Perray-en-Yvelines cedex (FR)

(21) Appl. No.: 11/097,666

(22) Filed: Apr. 1, 2005

(51) Int. Cl. A61Q 5/12; A61K 8/00; A61K 8/18

(52) U.S. Cl. 424/70.12; 424/70.1; 424/401; 424/47

(57) ABSTRACT

The present invention includes 1) a novel formulation for the treatment of hair loss comprising oleanolic acid (a 5α-reductase inhibitor), apigenin (a vasodilator), and biotinyl-GHK (a cell metabolism stimulant), 2) a novel additive for the treatment of hair loss comprising oleanolic acid, apigenin, biotinyl-GHK and a delivery agent, 3) a personal care, cosmetic, and/or dermopharmaceutical composition comprising oleanolic acid, apigenin, biotinyl-GHK, and at least one additional ingredient, and 4) a method for treating hair loss comprising the administration of oleanolic acid, apigenin, and biotinyl-GHK.

### BACKGROUND OF THE INVENTION

Each hair is formed at the level of a dermal papilla, which yields a hair bulb then a hair proper, through the cell division of keratinocytes. Obeying an internal clock, each papilla, located at the base of the hair follicle, receives a growth message necessary to trigger the cycle of natural renewal of the hair.

The hair cycle consists of three phases. The first phase, or growth phase, is known as anagen and lasts, on average, between three and four years. The second phase consists of discontinuation of growth over a period of two to three weeks. This phase is called catagen. The last phase, called telogen, is the phase when hair falls out. This phase occurs fairly slowly, over the course of three to four months, as the bulbar zone of hair follicle regresses and the hair shaft detaches and is expulsed towards the surface of the skin.

Hair cells are formed by the interaction between the dermis and the epidermis. An epidermal message stimulates the fibroblasts to organize and forward a signal to keratinocytes which induces formation of an epidermal plate which in turn invaginates in the dermis to form a primary bud. The primary bud emits messages which stabilize the surrounding fibroblasts to form the future dermal papilla. The bud gradually differentiates into a hair follicle under the influence of messages sent by the dermal papilla.

As in many systems in the human body, the chemical pathways and precise roles involved in the growth of hair are not fully known. However, it is known that hair growth is facilitated through the physical interaction between the dermis and epidermis within the dermal papilla wherein keratinocytes and fibroblasts are condensed. The dermal papilla is a zone that is particularly rich in collagens and glycosaminoglycans which maintain the close contact between the two cell populations and promote the chemical communication necessary for hair shaft growth. Collagen IV and laminin 5 are important proteoglycans as they constitute the basement membrane, the attachment zone for the epidermis and dermis, and, in the case of hairs, between the root sheath and the dermis.

Various forms of hair loss exist in both men and women and the present invention can be used to treat this hair loss. The most common type of hair loss is androgenetic alopecia, or male pattern baldness. This condition affects millions of people, roughly forty million in the United States alone. Male pattern baldness is generally caused by the transformation of testosterone into dihydrotestosterone which shrinks the size of hair follicles and the blood vessels that support them, eventually causing some of the follicles to shut down or even die. The transformation of testosterone into dihydrotestosterone is caused by the hormone, 5α-reductase. It is also known that deficiencies in vitamin H (biotin) can result in fine alopecic hair.

### SUMMARY OF THE INVENTION

Without wishing to be bound by any particular theory of operation, reducing levels of 5α-reductase will in turn reduce levels of dihydrotestosterone and thereby slow, prevent, or even reverse hair loss, particularly in men. The present invention includes formulations and methods for addressing this issue. The present invention comprises oleanolic acid, apigenin, biotinyl-GHK as active ingredients. In a preferred embodiment, one, two or all three of these active ingredients are provided in a single formulation. Most preferably, in one embodiment, the present invention comprises a single formulation including oleanolic acid, apigenin, biotinyl-GHK as active ingredients. In another embodiment, any one of the aforementioned formulations may also include at least one additional ingredient and/or delivery agent. Thus, in a particularly preferred embodiment, a formulation in accordance with the present invention includes oleanolic acid, apigenin, biotinyl-GHK as active ingredients and at least one additional ingredient and/or delivery agent. In addition or in the alternative to the additional ingredients, the formulations may include additional active ingredients.

In another embodiment, the present invention provides an additive for use in cosmetic and/or dermopharmaceutical formulations. The additive comprises (comprising oleanolic acid, biotinyl-GHK, apigenin and at least one delivery agent.

The delivery agent is generally selected and provided in an amount which is sufficient to solubilize, disburse, suspend, chelate, preserve, stabilize, structure, adjust the pH of, or protect from microbial attack the formulation comprising oleanolic acid, biotinyl-GHK and apigenin. These additives can then be used in formulating personal care products, cosmetics, and dermopharmaceuticals particularly those useful for reducing hair loss. In another embodiment, the additive consists essentially of oleanolic acid, biotinyl-GHK and apigenin and at least one delivery agent.

In a further embodiment, the present invention provides a personal care, cosmetic and/or dermopharmaceutical product comprising oleanolic acid, biotinyl-GHK, apigenin and at least one additional ingredient. Said personal care, cosmetic, and/or dermopharmaceutical composition may be in the form of a lotion, a shampoo, a conditioner, a hair spray, a gel, a hair styling product, a hair holding product, a sunscreen, a sunblock, a soap, a cream, an emulsion, a dispersion, a solution, a milk, a suspension, a cleanser, a wash, a scalp treatment lotion, and/or a spray.

The present invention further includes methods of treating hair loss in a subject in need of such a treatment comprising the steps of administering to at least one first portion of the scalp of said subject an effective amount of oleanolic acid, an effective amount of apigenin, and an effective amount of biotinyl-GHK. These active agents may be administered individually or in any possible combination. This administration could be in the form of a cosmetic or dermopharmaceutical composition.

The present invention aims to slow, stop, or even reverse hair loss. Without wishing to be bound by any particular theory of operation, this can be accomplished by one or more of: 1) reducing production of dihydrotestosterone by inhibiting 5α-reductase, 2) increasing blood flow to the dermis and thus to the hair follicles, and/or 3) ensuring better rooting of the hair in the skin.

### DETAILED DESCRIPTION

The use of oleanolic acid in the present invention was selected for its ability to inhibit 5α-reductase. The use of apigenin in the present invention was selected for its vasodilative properties. The use of biotinyl-GHK in the present invention was selected for its ability to improve hair adhesion by increasing the quality and duration of the anagen phase through improving the skin matrix and improving metabolic activity. Furthermore, enhancing anchorage of the hair sheath and dermis and increasing the length of the anagen phase should result from the use of biotinyl-GHK thereby delaying the onset of the telogen phase. Thus, the use of these three active ingredients can provide results which are not obtainable by the use of only two of them.

It has been discovered that oleanolic acid is endowed with a strong inhibitory action on the enzyme, 5α-reductase, and thus constitutes an important component for the treatment of hair loss. It has been discovered that oleanolic acid may thus be advantageously used in the treatment of hair loss, since oleanolic acid inhibits the transformation of testosterone into dihydrotestosterone.

Apigenin is known to be an effective vasodilator. It has been discovered however, that its use on the scalp dilates the blood vessels in the dermis which allows for a greater flow of blood to the hair follicles, promoting healthy hair growth. This makes apigenin ideal for use in treating hair loss.

Biotinyl-GHK has been found to have protective and reparative effects on the constituents of the root sheath and dermal papilla; collagen IV and laminin 5. It has been found to enhance structuring by increasing concentrations of adhesion proteins responsible for anchorage of the hair in the dermis, to enhance maintenance of a viable root sheath, and to have anti-aging activity on hair follicle keratinocytes. This makes biotinyl-GHK ideal for use in treating hair loss.

Because of the discoveries identified herein, the formulation of the present invention includes the selection of two of the three active ingredients identified herein such as use of biotinyl-GHK and apigenin, biotinyl-GHK and oleanolic acid, and oleanolic acid and apigenin. These may be formulated in a single formulation and used as such or may be applied separately to the same area of the scalp. The use of the third active in conjunction with the other two is particularly preferred. This can be separately formulated from the other two and applied to the same parts of the scalp, or they may be mixed just prior to application. However, in a particularly preferred aspect of the present invention, a single forumulation is made including apigenin, biotinyl-GHK and oleanolic acid, most preferably with at least one additional ingredient.

Any oleanolic acid may be used in the present invention at any purity. However, it is preferred that the oleanolic acid be at least eighty five percent pure and more preferably ninety-seven percent pure. It is also preferred that the oleanolic acid be obtained in the form of a titrated olive tree (Olea europaea) leaf extract.

Oleanolic acid can be obtained from many different plant species using any state of the art plant extraction method. It is preferred that oleanolic acid be extracted from olive tree leaves. The especially preferred species of olive tree is Olea europaea. Oleanolic acid is extracted from the leaves of this tree by solid/liquid extraction with ethanol. This is followed by concentration of the ethanol, filtration and drying. It may be possible to obtain a yield of 90% of the leaf's oleanolic acid content in a purity of 95% after only a single extraction stage.

Oleanolic acid is one of hundreds of diterpenes. It is contemplated that other diterpens/sapogenins may be substituted for the oleanolic acids. Specifically it may be possible to substitute ursolic acid, beta-amyrin, alpha-amyrin, quillaic acid, Asiatic acid, eriantic acid, taraxerol. Stereoisomers of oleanolic acid, such as epi-oleanolic acid, an also be used. Additionally glucoside or ester derivatives of oleanolic acid may be used.

Biotinyl-GHK is a chemical with the general structure: 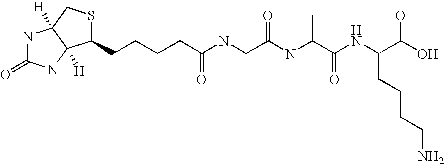
Biotinyl-GHK is also known by the names biotinyl tripeptide and Biotinyl glycyl-hystidyl-lysine. Biotinyl-GHK can be formed by binding the peptide Glycyl-Hystidyl-Lysine and vitamin H (biotin). This can be done by conventional chemical synthesis in heterogenous phase or homogeneous phase as disclosed in J. M. Stewart, Solid Phase Peptide Synthesis, and J. D. Young, ISBN 0-935940-03-0, Ed. 2 (Pierce Chemical Company 1984) which are hereby incorporated by reference, or by enzymatic synthesis, as disclosed in Kulman et a., J. Biol. Chem. 255, 8234 (1980) which is hereby incorporated by reference, from constituent amino acids or their derivatives. It is preferable for biotinyl-GHK to be synthesized by stepwise peptide synthesis. The C terminal of the aminoacid (lys) is protected on its acidic function, then each protected amino acid (Glu, His) is successively coupled by standard, state of the art, amid bond formation, as described in French Patent 2,791,684, which is hereby incorporated by reference, or standard texts on peptid synthesis. Finally, a last coupling procedure is performed with biotin (Vitamin H), instead of an amino acid, and the protected peptide is cleaved to remove all protecting groups. This coupling takes place in a basic environment that is preferably anhydrous. It is preferable for the grafting of biotin on free amine functions of a peptide or a peptide derivative, protected or not, be made by reacting esters derived from biotin (i.e. paranitrophenyl ester or N-hydroxy-succinimid ester), or by any other activation form of biotin (i.e. EtOCOCl, DCC, TBTU, BOP). Following the coupling the product can be purified by classical methods of peptide chemistry, such as, crystallization and chromatography. The grafting can also be made directly during the solid phase synthesis of the peptide, as disclosed in Lobl et al., Anal. Biochem., 170, 502-505 (1988) which is hereby incorporated by reference. In that case, an excess of the reagent is used and the coupling time is increased to compensate for the lowest reactivity. The last step of the peptide cleavage is not modified because of the presence of the biotin on the peptide.

Any grade or purity of biotinyl-GHK can be used but it is preferable that such purity be at least 75%. Additionally, various salts of biotinyl-GHK are also acceptable for use, including the acetate, chlorhydrate, and trifluoroacetate salts. It is also contemplated that other GHK derivatives could be used.

Apigenin is a nonmutagenic bioflavonoid which is present in many types of plants and vegetables including, but not limited to, grapefruit, parsley, chamomile, apples, celery, basil, oregano, tarragon, cilantro, parsley, passion flower. The preferred method for producing the apigenin used in the present invention includes extracting the apigenin from ground citrus fruits with a methanol/water extraction.

Various purities of apigenin can be used but it is preferred that the apigenin be at least 75% pure and more preferably 90% pure.

Oleanolic acid should be administered to the scalp of a subject in an amount no less than 0.0000000003 mg per cm² of the scalp to be effective. More preferably, no less than 0.0000003 mg per cm² of oleanolic acid should be administered to the scalp. Biotinyl-GHK should be administered to the scalp of a subject in an amount no less than 0.0000000002 per cm² of the scalp to be effective. More preferably, no less than 0.0000002 mg per cm² of biotinyl-GHK should be applied to the scalp. Apigenin should be administered to the scalp of a subject in an amount no less than 0.0000000005 per cm² of the scalp to be effective. More preferably, no less than 0.0000005 mg per cm² of apigenin should be applied to the scalp.

Any amount of each of the three actives (oleanolic acid, apigenin, and biotinyl-GHK) can be present in proportion to one another so long as there is at least some (0.0001% or more) of each of oleanolic acid, apigenin, and biotinyl-GHK. Thus, the relative proportions could be, for example only, 0.0001% oleanolic acid, 0.0001% apigenin and 99.9998% biotinyl-GHK. However, the amount of each active present should be such that when formulated in a personal care, cosmetic, and/or dermopharmaceutical product, it is possible to provide an effective amount of each, preferably in a reasonable volume. Thus, it is advantageous to have, significant preportions of each of the three actives, such as, just for purposes of example, 50% apigenin, 30% oleanolic acid, and 20% biotinyl-GHK. Preferably the each acitive is present in an amount of between 1% and 80% by weight and more preferably between about 15% and 60% by weight.

In accordance with the present invention, the term “additive” is distinguished from formulations, personal care, cosmetic, or dermopharmaceutical compounds. An additive in accordance with the present invention includes oleanolic acid, biotinyl-GHK and apigenin, as described herein, in combination with certain delivery agents which are useful in performing certain various functions in support of the formulation. These functions include, without limitation, solubilizing, dispersing, suspending, structuring, chelating, preserving, stabilizing, adjusting the pH of or protecting from antimicrobial attack the formulation (that comprising oleanolic acid, biotinyl-GHK, and apigenin).

Generally these additives are not intended to be applied directly to the hair or skin in place of a personal care, cosmetic, or dermopharmaceutical composition, preparation, or formulation. Instead, these additives may be supplied to those who manufacture such personal care, cosmetic or dermopharmaceutical compositions. From these additives, manufacturers may remove the combination of oleanolic acid, aginenin, and biotinyl-GHK such as by evaporating any included solvent, adding only the formulation to, for example, their personal care composition. Alternatively, the additive can be measured out so as to provide a sufficient amount of the oleanolic acid, aginenin, and biotinyl-GHK for a given preparation and that portion of the additive used as one component in such personal care product. An additive, in accordance with the invention, therefore encompasses something, at least some portion of which, is added to a personal care product but is not itself a personal care product.

Certain additives in accordance with the present invention can be characterized by the phrase “consisting essentially of.” This term is used herein to exclude those things which would materially alter the basic and novel characteristics of the additives. For example, consider a personal care product that will include a chelating agent as well as an additive containing a chelating agent. A chelating agent would not be something which is, in and of itself, contrary to the basic and novel aspects of the invention. However, the amount of chelating agent found in the additive should be sufficient to provide adequate chelation for the formulation and/or any other reason to the quality and function of the additive. A suitable excess may also be present. However, in general, the amount of chelating agent present in the additive will not be predicated on the amount necessary for use in the final formulation. If sufficient chelating agent is contained in the additive, that would be a matter of serendipity.

Materials such as humectants, emollients, fragrances and colorings would typically not be included within the phrase “consisting essentially of” unless they served some purpose of enhancing solubility, dispersion, stability, handling or the like of the additive per se. In those instances, they would only be present in an amount which is sufficient to provide those properties and a reasonable excess as appropriate.

The oleanolic acid, apigenin, and biotinyl-GHK, can be present in the additive in any amounts that would give an effective amount to any subsequent personal care, cosmetic, and/or dermopharmaceutical composition as defined above with the remainder of the additive comprising the at least one delivery agent. The concentration of the oleanolic acid, aginenin, and biotinyl-GHK present in the additive depends on many considerations, including, but not limited to, the intended use of the additive, the amount of additive to be included, the methods used in preparing the additive, the number and types of delivery agents used, the number and types of other additives used in the personal care, cosmetic, and/or dermopharmaceutical composition, and the type and intended use of the personal care, cosmetic, and/or dermopharmaceutical composition.

It is most convenient to discuss the three actives present as a group. As such, an additive in the context of the present invention comprises an active (which is in reality oleanolic acid, apigenin, and biotinyl-GHK in any proportions previously discussed) and one or more delivery agents, which for convenience, are also discussed as a group. Thus, for an additive, the amount of active may be any amount greater than zero, with the balance being delivery agent. Preferably, the additive will have sufficient active to allow for preparation of personal care, cosmetic, and/or dermopharmaceutical compositions of a convenient volume having at least an effective amount of the oleanolic acid, apigenin, and biotinyl-GHK. Preferably the combined actives will be present in an amount of at least about 0.001% by weight, more preferably between about 0.01% and 0.1% by weight. The balance will be delivery agent.

The additive discussed above can be incorporated as an additive in personal care, cosmetic, or dermopharmaceutical compositions. When a personal care, cosmetic, and/or dermopharmaceutical composition according to the present invention is prepared with the actives present in the form of an additive, the amount of additive used will depend on a number of factors, as discussed above. Significant factors affecting the amount of additive used in the composition depend on how much active is to be included in the composition and how concentrated the level of active is in the additive. Preferably the additive is included at a level between about 0.01% and 25%, more preferably between 0.05% and 10%, and most preferably between about 0.1 and 5% by weight.

The present invention also includes personal care, cosmetic, and/or dermopharmaceutical compositions comprising oleanolic acid, apigenin, and biotinyl-GHK. The actives present in the personal care, cosmetic, and/or dermopharmaceutical composition of the present invention can be incorporated into the composition as an additive or can be added individually into the composition. When the actives are added individually they are added in accordance with the levels discussed above with respect to adding the additive. In such personal care, cosmetic and/or dermopharmaceutical compositions, oleanolic acid must be applied to the scalp of a subject in an amount no less than about 0.0000000003 mg/cm² to be effective in treating hair loss, apigenin must be applied to the scalp of said subject in amount no less than about 0.0000000005 mg/cm² to be effective in treating hair loss, and biotinyl-GHK must be applied to the scalp of said subject in an amount no less than about 0.0000000002 mg/cm² to be effective in treating hair loss.

In personal care, cosmetic, or dermopharmaceutical compositions, it is unnecessary, but particularly advantageous to combine oleanolic acid, apigenin, and biotinyl-GHK with or without other active substances in order to strengthen their overall effect. Such other active substances may include ANCRINE™ (a complex of wheat protein and octylbutyrate which promotes hair anchoring), CAPILECTINE (a potato derived growth factor protein which stimulates hair bulb activity), CAPIGEN (a complex composed of homotaurine, chondroitine sulphate and a proprietary bioferment which also reduces hair loss), Minoxidil, Aminexil, Finasteride, Glucosamine, GABA, aminopropane phosphonic acid, matrikine peptides (including di, tri, tetra-, penta-, hexa-, etc. peptides), and genistein, daidzeine and similar isoflavones from soy or red clover.

Preferred additional ingredients in the personal care, cosmetic, and/or dermopharmaceutical compositions of the present invention include vitamins, especially preferable are vitamins B and PP, panthenol (vitamin B5), and tocopherol acetate, alcohol, peptides, scalp cleansers, stimulators of hair growth, improvers of keratin biosythesis, circulation activators, tonifying agents, fortifying agents, essential oils, soothing agents, surfactants, niacinamide, pyridoxine, DMDM hydantoin hydrolysed soy proteins, ornithine, chlorphenesin, arginine, organic silicium, chelating agents, glycols, and antioxidants.

According to the present invention “formulation” is a generic term which can encompass additives, personal care, cosmetic, and dermopharmaceutical compositions, and in situe coreations of additives and personal care, cosmetic, and dermopharmaceutical compositions. Formulations can also cover additives and personal care, cosmetic and dermopharmaceutical compositions comprising any one or two of oleanolic acid, apigenin, and biotinyl-GHK to which is added the remaining one or two of the three actives. For example, a formulation would cover a cosmetic composition containing oleanolic acid and apigening to which biotinyl-GHK is subsequently added. Such an addition can be done at any time prior to application or even where the three actives are applied separately and mixed together on the scalp.

Oleanolic acid, apigenin, and biotinyl-GHK, together or combined with other active substances, may be used in any form employed in cosmetics or dermopharmacy: such as lotions, shampoos, conditioners, hair sprays, gels, hair styling products, hair holding products, sunscreens, sunblocks, soaps, creams, emulsions, dispersions, solutions, milks, suspensions, cleansers, washes, scalp treatment lotions, or sprays.

Oleanolic acid, apigenin, and biotinyl-GHK, together or combined with other active substances, and or combined with at least one additional ingredient and/or delivery agent, may be used in the form of solutions, dispersions and emulsions, or encapsulated in carriers such as macrocapsules, microcapsules or nanocapsules, and macrospheres, microspheres or nanospheres, and liposomes, oleosomes or chylomicrons, or included in macroparticles, microparticles or nanoparticles or in macrosponges, microsponges or nanosponges, or adsorbed on powdered organic polymers, talcs, bentonites or other inorganic carriers.

Oleanolic acid, apigenin, and biotinyl-GHK, may be combined with any additional ingredient which may be active, functional, conventionally used in cosmetic, personal care or topical/transdermal pharmaceutical products or otherwise. Of course, a decision to include an additional ingredient and the choice of specific additional ingredients depends on the specific application and product formulation. Also, the line of demarcation between an “active” ingredient and an “inactive ingredient” is artificial and dependent on the specific application and product type. A substance that is an “active” ingredient in one application or product may be a “functional” ingredient in another, and vice versa. A particular ingredient might provide substantivity in one formulation, facilitate transdermal application in another, and merely provide proper viscosity in a third. Which of these is functional and which is active is subject to debate. But, regardless of the outcome, the material in question would qualify as an additional ingredient in accordance with the present invention.

Thus, the compositions of the invention may include one or more additional ingredients, which provide some benefit to the object of the composition. Such additional ingredients may include one or more substances such as, without limitations, cleaning agents, surfactants, hair conditioning agents, skin conditioning agents, hair styling agents, antidandruff agents, hair growth promoters, sunscreen and/or sunblock compounds for hair and/or skin, moisturizers/humectants, film formers, thickening agents, emulsifiers, emollients, dermatologically acceptable carriers. In a preferred embodiment, where the composition is to be in contact with human keratinous scalp tissue and/or hair, the additional ingredients should be suitable for application to keratinous scalp tissue and/or hair, that is, when incorporated into the composition they are suitable for use in contact with human keratinous scalp tissue and/or hair without undue toxicity, incompatibility, instability, allergic response, and the like within the scope of sound medical judgment. The CTFA Cosmetic Ingredient Handbook, Tenth Edition (Cosmetic, Toiletry & Fragrance Association 2004) describes a wide variety of nonlimiting cosmetic and pharmaceutical ingredients commonly used in the skin care industry, which are suitable for use as additional ingredients in the compositions of the present invention. Non-limiting examples of these additional ingredient classes include: aesthetic components such as fragrances, colorings/colorants, essential oils, astringents, etc. (e.g., clove oil, menthol, camphor, eucalyptus oil, eugenol, menthyl lactate, witch hazel distillate), antimicrobial agents antioxidants, binders, biological additives, buffering agents, chelating agents, chemical additives, colorants, cosmetic astringents, cosmetic biocides, denaturants, film formers or materials, e.g., polymers, for aiding the film-forming properties and substantivity of the composition opacifying agents, pH adjusters, propellants, reducing agents, sequestrants, skin-conditioning agents skin soothing and/or healing agents (e.g., panthenol and derivatives aloe vera, pantothenic acid and its derivatives, allantoin, bisabolol, and dipotassium glycyrrhizinate), skin treating agents, thickeners, and vitamins and derivatives thereof. More particularly, additional ingredients include glycerol, sorbitol, pentaerythritol, pyrrolidone acid and its salts, sunscreens; plant tissue extracts, polysaccharide; anti-dandruff agents; antiseborrheic agents, an oxidant, a bleaching agent, a reducing agent, a vitamin, a steroid, an enzyme, a non-steroidal anti-inflammatory, an an antimicrobial, substances intended to improve the state of dry or aged skin, tocopherols, vitamins E, F or A and their esters, antioxidants, essential fatty acids, glycyrrhetinic acid, keratolytics and carotenoids, ceramides and pseudo-ceramides, and all lipid complexes of a form similar to that of the natural ceramides of the scalp.

In any embodiment of the present invention, however, the additional ingredients useful herein can be categorized by the benefit they provide or by their postulated mode of action. However, it is to be understood that the additional ingredients useful herein can in some instances provide more than one benefit or operate via more than one mode of action. Therefore, classifications herein are made for the sake of convenience and are not intended to limit the additional ingredients to that particular application or applications listed.

Farnesol

The topical compositions of the present invention may contain a safe and effective amount of farnesol. Farnesol is a naturally occurring substance which is believed to act as a precursor and/or intermediate in the biosynthesis of squalene and sterols, especially cholesterol. Farnesol is also involved in protein modification and regulation (e.g., farnesylation of proteins), and there is a cell nuclear receptor which is responsive to farnesol.

Chemically, farnesol is [2E,6E]-3,7,11-trimethyl-2,6,10-dodecatrien-1-ol and as used herein “farnesol” includes isomers and tautomers of such. Farnesol is commercially available, e.g., under the names farnesol (a mixture of isomers from Dragoco, 10 Gordon Drive, Totowa, N.J.) and trans-trans-farnesol (Sigma Chemical Company, P.O. Box 14508, St. Louis, Mo.).

When present in the compositions of the present invention, the composition preferably contains from about 0.001% to about 50%, by weight of the composition, more preferably from about 0.01% to about 20%, even more preferably from about 0.1% to about 15%, even more preferably from about 0.1% to about 10%, still more preferably from about 0.5% to about 5%, and still more preferably from about 1% to about 5% of farnesol.

Phytantriol

The topical compositions of the present invention may contain a safe and effective amount of phytantriol. Phytantriol is the common name for the chemical known as 3,7,11,15, tetramethylhexadecane-1,2,3,-triol. Phytantriol is commercially available from BASF (1609 Biddle Avenue, Whyandotte, Mich.). For example, phytantriol is useful as a spider vessel/red blotchiness repair agent, a dark circle/puffy eye repair agent, sallowness repair agent, a sagging repair agent, an anti-itch agent, a skin thickening agent, a pore reduction agent, oil/shine reduction agent, a post-inflammatory hyperpigmentation repair agent, wound treating agent, an anti-cellulite agent, and regulating skin texture, including wrinkles and fine lines.

When present in the compositions of the present invention, the composition preferably contains from about 0.001% to about 50%, by weight of the composition, more preferably from about 0.01% to about 20%, even more preferably from about 0.1% to about 15%, even more preferably from about 0.1% to about 10%, still more preferably from about 0.5% to about 5%, and still more preferably from about 1% to about 5% of farnesol.

In the compositions of the present invention, the phytantriol preferably is included in an amount from about 0.001% to about 50% by weight of the composition, more preferably from about 0.01% to about 20%, even more preferably from about 0.1% to about 15%, even more preferably from about 0.2% to about 10%, still more preferably from about 0.5% to about 10%, and still more preferably from about 1% to about 5%.

Desquamation Actives

A safe and effective amount of a desquamation active may be added to the compositions of the present invention, more preferably from about 0.1% to about 10%, even more preferably from about 0.2% to about 5%, also preferably from about 0.5% to about 4%, by weight of the composition. Desquamation actives enhance the skin appearance benefits of the present invention. For example, the desquamation actives tend to improve the texture of the skin (e.g., smoothness). One desquamation system that is suitable for use herein contains sulfhydryl compounds and zwitterionic surfactants and is described in U.S. Pat. No. 5,681,852, to Bissett, incorporated herein by reference. Another desquamation system that is suitable for use herein contains salicylic acid and zwitterionic surfactants and is described in U.S. Pat. No. 5,652,228 to Bissett, incorporated herein by reference. Zwitterionic surfactants such as described in these applications are also useful as desquamatory agents herein, with cetyl betaine being particularly preferred.

a) Vitamin B3 Compounds

The compositions of the present invention may contain a safe and effective amount of a vitamin B3 compound. Vitamin B3 compounds are particularly useful for regulating skin condition as described in co-pending U.S. application Ser. No. 08/834,010, filed Apr. 11, 1997 (corresponding to international publication WO 97/39733 A1, published Oct. 30, 1997). When vitamin B3 compounds are present in the compositions of the instant invention, the compositions preferably contain from about 0.01% to about 50%, more preferably from about 0.1% to about 10%, even more preferably from about 0.5% to about 10%, and still more preferably from about 1% to about 5%, still more preferably from about 2% to about 5%, by weight of the composition, of the vitamin B3 compound.

As used herein, “vitamin B3 compound” means a compound having the formula: 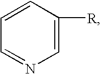
wherein R is —CONH₂ (i.e., niacinamide), —COOH (i.e., nicotinic acid) or —CH₂OH (i.e., nicotinyl alcohol); derivatives thereof; and salts of any of the foregoing.

Exemplary derivatives of the foregoing vitamin B3 compounds include nicotinic acid esters, including non-vasodilating esters of nicotinic acid (e.g., tocopheryl nicotinate), nicotinyl amino acids, nicotinyl alcohol esters of carboxylic acids, nicotinic acid N-oxide and niacinamide N oxide.

Examples of suitable vitamin B3 compounds are well known in the art and are commercially available from a number of sources, e.g., the Sigma Chemical Company (St. Louis, Mo.); ICN Biomedicals, Inc. (Irvin, Calif.) and Aldrich Chemical Company (Milwaukee, Wis.).

The vitamin compounds may be included as the substantially pure material, or as an extract obtained by suitable physical and/or chemical isolation from natural (e.g., plant) sources.

c) Hydroxy Acids

The compositions of the present invention may contain a safe and effective amount of a rimeste acid. Preferred rimeste acids for use in the compositions of the present invention include salicylic acid and salicylic acid derivatives. When present in the compositions of the present invention, salicylic acid is preferably used in an amount of from about 0.01% to about 50%, more preferably from about 0.1% to about 20%, even more preferably from about 0.1% to about 10%, still more preferably from about 0.5% to about 5%, and still more preferably from about 0.5% to about 2%.

Anti-Oxidants/Radical Scavengers

The compositions of the present invention may include a safe and effective amount of an anti-oxidant/radical scavenger or an oxidizer/reducing agent. The anti-oxidant/radical scavenger or oxidizer/reducing agent is especially useful for providing protection against UV radiation which can cause increased scaling or texture changes in the stratum corneum and against other environmental agents which can cause skin damage. These compounds may also be useful in hair drying and other cosmetic applications.

A safe and effective amount of an anti-oxidant/radical scavenger or an oxidizer/reducing agent may be added to the compositions of the subject invention, preferably from about 0.1% to about 10%, more preferably from about 1% to about 5%, of the composition.

Anti-oxidants/radical scavengers such as ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated Irimeste benzoic acids and their salts, peroxides including hydrogen peroxide, perborate, thioglycolates, persulfate salts, 6-hydroxy-2,5,7,8-tetramethylchroman-2-carboxylic acid (commercially available under the tradename Trolox®), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, 1-methionine, Irimest, superoxide dismutase, silymarin, tea extracts, grape skin/seed extracts, melanin, and rosemary extracts may be used. Preferred anti-oxidants/radical scavengers are selected from tocopherol sorbate and other esters of tocopherol, more preferably tocopherol sorbate. For example, the use of tocopherol sorbate in topical compositions and applicable to the present invention is described in U.S. Pat. No. 4,847,071, issued on Jul. 11, 1989 to Donald L. Bissett, Rodney D. Bush and Ranjit Chatterjee. Especially useful are combinations with the antioxidant enzymes called VENUCEANE® offered by SEDERMA, described in WO 02/066668 published on Aug. 29, 2002.

Chelators

The compositions of the present invention may also contain a safe and effective amount of a chelator or chelating agent. As used herein, “chelator” or “chelating agent” means an active agent capable of removing a metal ion from a system by forming a complex so that the metal ion cannot readily participate in or catalyze chemical reactions. The inclusion of a chelating agent is especially useful for providing protection against UV radiation which can contribute to excessive scaling or skin texture changes and against other environmental agents which can cause skin damage.

A safe and effective amount of a chelating agent may be added to the compositions of the subject invention, preferably from about 0.1% to about 10%, more preferably from about 1% to about 5%, of the composition. Exemplary chelators that are useful herein are disclosed in U.S. Pat. No. 5,487,884, issued Jan. 30, 1996 to Bissett et al.; International Publication No. 91/16035, Bush et al., published Oct. 31, 1995; and International Publication No. 91/16034, Bush et al., published Oct. 31, 1995. Preferred chelators useful in compositions of the subject invention are furildioxime, furilmonoxime, and derivatives thereof.

Anti-Inflammatory Agents

A safe and effective amount of an anti-inflammatory agent may be added to the compositions of the present invention, preferably from about 0.1% to about 10%, more preferably from about 0.5% to about 5%, of the composition. The anti-inflammatory agent enhances the skin appearance benefits of the present invention, e.g., such agents contribute to a more uniform and acceptable skin tone or color. The exact amount of anti-inflammatory agent to be used in the compositions will depend on the particular anti-inflammatory agent utilized since such agents vary widely in potency.

Steroidal anti-inflammatory agents, including but not limited to, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. The preferred steroidal anti-inflammatory for use is hydrocortisone.

A second class of anti-inflammatory agents which is useful in the compositions includes the nonsteroidal anti-inflammatory agents. The variety of compounds encompassed by this group are well-known to those skilled in the art. For detailed disclosure of the chemical structure, synthesis, side effects, etc. of non-steroidal anti-inflammatory agents, one may refer to standard texts, including K. D. Rainsford, Anti-inflammatory and Anti-Rheumatic Drugs, Vol. I-III, (CRC Press 1985), and Anti-inflammatory Agents, Chemistry and Pharmacology, 1, R. A. Scherrer, et al., (Academic Press 1974).

Specific non-steroidal anti-inflammatory agents useful in the composition invention include, but are not limited to:
1) the oxicams, such as piroxicam, isoxicam, tenoxicam, sudoxicam, and CP-14,304;
2) the salicylates, such as aspirin, disalcid, benorylate, trilisate, safapryn, solprin, diflunisal, and fendosal;
3) the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac;
4) the fenamates, such as mefenamic, meclofenamic, flufenamic, niflumic, and tolfenamic acids;
5) the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and
6) the pyrazoles, such as phenylbutazone, oxyphenbutazone, feprazone, azapropazone, and trimethazone.

Mixtures of these non-steroidal anti-inflammatory agents may also be employed, as well as the dermatologically acceptable salts and esters of these agents. For example, etofenamate, a flufenamic acid derivative, is particularly useful for topical application. Of the nonsteroidal anti-inflammatory agents, ibuprofen, naproxen, flufenamic acid, etofenamate, aspirin, mefenamic acid, meclofenamic acid, piroxicam and felbinac are preferred; ibuprofen, naproxen, ketoprofen, etofenamate, aspirin and flufenamic acid are more preferred.

Finally, so-called “natural” anti-inflammatory agents are useful in methods of the present invention. Such agents may suitably be obtained as an extract by suitable physical and/or chemical isolation from natural sources (e.g., plants, fungi, by-products of microorganisms) or can be synthetically prepared. For example, candelilla wax, bisabolol (e.g., alpha bisabolol), aloe vera, plant sterols (e.g., phytosterol), Manjistha (extracted from plants in the genus Rubia, particularly Rubia Cordifolia), and Guggal (extracted from plants in the genus Commiphora, particularly Commiphora Mukul), kola extract, chamomile, red clover extract, Piper methysticum extract (Kava Kava from SEDERMA, disclosed in FR 2 771 002 of Mar. 31, 2000 and WO 99/25369 published on May 27, 1999), Bacopa monieri extract (Bacocalmine from SEDERMA, disclosed in WO 99/40897 of Aug. 19, 1999) and sea whip extract, may be used.

Additional anti-inflammatory agents useful herein include compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C₂-C₂₄ saturated or unsaturated esters of the acids, preferably C₁₀ C₂₄, more preferably C₁₆-C₂₄. Specific examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate. Stearyl glycyrrhetinate is preferred.

Skin Soothing and Skin Healing Actives

The compositions of the present invention may comprise a skin soothing or skin healing active. Skin soothing or skin healing actives suitable for use herein include panthenoic acid derivatives (including panthenol, dexpanthenol, ethyl panthenol), aloe vera, allantoin, bisabolol, and dipotassium glycyrrhizinate. A safe and effective amount of a skin soothing or skin healing active may be added to the present composition, preferably, from about 0.1% to about 30%, more preferably from about 0.5% to about 20%, still more preferably from about 0.5% to about 10%, by weight of the composition formed. Especially useful are combinations with the skin soothing and healing agents called Calmosensine and Bacocalmine offered by SEDERMA and described in WO 98/07744 of Feb. 26, 1998 and WO 99/40897 of Aug. 19, 1999 respectively.

Bisabolol

The topical compositions of the present invention may also contain a safe and effective amount of bisabolol. Bisabolol is a naturally occurring unsaturated monocyclic terpene alcohol having the following structure: 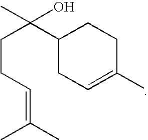
It is the primary active component of chamomile extract/oil. Bisabolol can be synthetic (d,1-alpha-isomer or (±)-alpha-isomer) or natural ((−)-alpha-isomer) in origin and can be used as essentially pure compounds or mixtures of compounds (e.g., extracts from natural sources such as chamomile). The alpha form of bisabolol (a-bisabolol) is used in a variety of cosmetic products as a skin conditioning or soothing agent. As used herein, “bisabolol” includes chamomile extract or oil and any isomers and tautomers of such. Suitable bisabolol compounds are commercially available as a natural material from Dragoco (Totowa, N.J.) under the product name alpha-bisabolol natural and as a synthetic material from Fluka (Milwaukee, Wis.) under the product name alpha-bisabolol.

In the compositions of the present invention, the composition preferably contains from about 0.001% to about 50%, by weight of the composition, more preferably from about 0.01% to about 20%, even more preferably from about 0.01% to about 15%, and still more preferably from about 0.1% to about 10%, of bisabolol, even more preferably from about 0.1% to about 5%.

Antimicrobial and Antifungal Actives

The compositions of the present invention may contain an antimicrobial or antifungal active. Such actives are capable of destroying microbes, preventing the development of microbes or preventing the pathogenic action of microbes. A safe and effective amount of an antimicrobial or antifungal active may be added to the present compositions, preferably, from about 0.001% to about 10%, more preferably from about 0.01% to about 5%, and still more preferably from about 0.05% to about 2%.

Examples of antimicrobial and antifungal actives include β-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, zinc pyrithione and clotrimazole. Especially useful are combinations with the ingredient range called OSMOCIDE offered by SEDERMA and described in WO 97/05856 of Feb. 20, 1997.

Preferred examples of actives useful herein include those selected from salicylic acid, benzoyl peroxide, 3-hydroxy benzoic acid, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, phytic acid, N-acetyl-L-cysteine, lipoic acid, azelaic acid, arachidonic acid, benzoylperoxide, tetracycline, ibuprofen, naproxen, hydrocortisone, rimesters en, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neocycin sulfate, and mixtures thereof.

Sunscreen Actives

Exposure to ultraviolet light can result in excessive scaling and texture changes of the stratum corneum. Therefore, the compositions of the subject invention may optionally contain a sunscreen active. As used herein, “sunscreen active” includes both sunscreen agents and physical sunblocks. Suitable sunscreen actives may be organic or inorganic.

Inorganic sunscreens useful herein include the following metallic oxides; titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof. When used herein, the inorganic sunscreens are present in the amount of from about 0.1% to about 20%, preferably from about 0.5% to about 10%, more preferably from about 1% to about 5%, by weight of the composition.

A wide variety of conventional organic sunscreen actives are suitable for use herein. Sagarin, et al., at Chapter VIII, pages 189 et seq., of Cosmetics Science and Technology (1972), discloses numerous suitable actives. Specific suitable sunscreen actives include, for example: p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); Irimeste- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene boman-2-one), terephthalylidene dicamphor sulfonic acid and 4-isopropyl-di-benzoylmethane.

Of these, 2-ethylhexyl-p-methoxycinnamate (commercially available as PARSOL MCX), 4,4′-t-butyl methoxydibenzoyl-methane (commercially available as PARSOL 1789), 2-hydroxy-4-methoxybenzophenone, octyldimethyl-p-aminobenzoic acid, digalloyltrioleate, 2,2-dihydroxy-4-methoxybenzophenone, ethyl-4-(bis( rimeste-propyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene and mixtures of these compounds, are preferred.

Also preferred are the compositions and combinations described and claimed in U.S. Pat. No. 6,190,645 to SaNogueira et al. and in particular, sunscreen agents disclosed at col. 3, lns. 4-23, in combination with a cinnamido alkyl amine cationic quaternary salt such as cinnamidopropyl trimethyl ammonium chloride sold under the trademark INCROQUAT-UV-283 manufactured by Croda, Inc., 7 Century Road, Parsippany, N.J. These portions of the U.S. Pat. No. 6,190,645 patent are herby incorporated by reference. More preferred organic sunscreen actives useful in the compositions useful in the subject invention are 2-ethylhexyl-p-methoxycinnamate, butylmethoxydibenzoyl-methane, 2-hydroxy-4-methoxybenzo-phenone, 2-phenylbenzimidazole-5-sulfonic acid, octyldimethyl-p-aminobenzoicacid, octocrylene and mixtures thereof.

Also particularly useful in the compositions are sunscreen actives such as those disclosed in U.S. Pat. No. 4,937,370 issued to Sabatelli on Jun. 26, 1990, and U.S. Pat. No. 4,999,186 issued to Sabatelli & Spirnak on Mar. 12, 1991. The sunscreening agents disclosed therein have, in a single molecule, two distinct chromophore moieties which exhibit different ultra-violet radiation absorption spectra. One of the chromophore moieties absorbs predominantly in the UVB radiation range and the other absorbs strongly in the UVA radiation range.

Preferred members of this class of sunscreening agents are 4-N,N-(2-ethylhexyl)methyl-aminobenzoic acid ester of 2,4-dihydroxybenzophenone; N,N-di-(2-ethylhexyl)-4-aminobenzoic acid ester with 4-hydroxydibenzoylmethane; 4-N,N-(2-ethylhexyl)methyl-aminobenzoic acid ester with 4-hydroxydibenzoylmethane; 4-N, N-(2-ethylhexyl)methyl-aminobenzoic acid ester of 2-hydroxy-4-(2-hydroxyethoxy)benzophenone; 4-N,N-(2-ethylhexyl)-methylaminobenzoic acid ester of 4-(2-hydroxyethoxy)dibenzoylmethane; N,N-di-(2-ethylhexyl)-4-aminobenzoic acid ester of 2-hydroxy-4-(2-hydroxyethoxy)benzophenone; and N,N-di-(2-ethylhexyl)-4-aminobenzoic acid ester of 4-(2-hydroxyethoxy)dibenzoylmethane and mixtures thereof.

Especially preferred sunscreen actives include 4,4′-t-butylmethoxydibenzoylmethane, 2-ethylhexyl-p-methoxycinnamate, phenyl benzimidazole sulfonic acid, and octocrylene.

A safe and effective amount of the organic sunscreen active is used, typically from about 1% to about 20%, more typically from about 2% to about 10% by weight of the composition. Exact amounts will vary depending upon the sunscreen or sunscreens chosen and the desired Sun Protection Factor (SPF).

Conditioning Agents

The compositions of the present invention may contain a conditioning agent selected from humectants, moisturizers, or skin conditioners. A variety of these materials can be employed and each can be present at a level of from about 0.01% to about 20%, more preferably from about 0.1% to about 10%, and still more preferably from about 0.5% to about 7% by weight of the composition. These materials include, but are not limited to, guanidine; urea; glycolic acid and glycolate salts (e.g. ammonium and quaternary alkyl ammonium); salicylic acid; lactic acid and lactate salts (e.g., ammonium and quaternary alkyl ammonium); aloe vera in any of its variety of forms (e.g., aloe vera gel); starches; sugar and starch derivatives (e.g., alkoxylated glucose, fructose, glucosamine); hyaluronic acid; lactamide monoethanolamine; acetamide monoethanolamine; panthenol; allantoin; and mixtures thereof. Also useful herein are the propoxylated glycerols described in U.S. Pat. No. 4,976,953, to Orr et al., issued Dec. 11, 1990.

Also useful are various C₁-C₃₀ monoesters and polyesters of sugars and related materials. These esters are derived from a sugar or polyol moiety and one or more carboxylic acid moieties. Such ester materials are further described in, U.S. Pat. No. 2,831,854, U.S. Pat. No. 4,005,196, to Jandacek, issued Jan. 25, 1977; U.S. Pat. No. 4,005,195, to Jandacek, issued Jan. 25, 1977, U.S. Pat. No. 5,306,516, to Letton et al., issued Apr. 26, 1994; U.S. Pat. No. 5,306,515, to Letton et al., issued Apr. 26, 1994; U.S. Pat. No. 5,305,514, to Letton et al., issued Apr. 26, 1994; U.S. Pat. No. 4,797,300, to Jandacek et al., issued Jan. 10, 1989; U.S. Pat. No. 3,963,699, to Rizzi et al., issued Jun. 15, 1976; U.S. Pat. No. 4,518,772, to Volpenhein, issued May 21, 1985; and U.S. Pat. No. 4,517,360, to Volpenhein, issued May 21, 1985.

Preferably, the conditioning agent is selected from urea, guanidine, sucrose polyester, panthenol, dexpanthenol, allantoin, and combinations thereof.

Structuring Agents

The compositions hereof, and especially the emulsions hereof, may contain a structuring agent. Structuring agents are particularly preferred in the oil-in-water emulsions of the present invention. Without being limited by theory, it is believed that the structuring agent assists in providing Theological characteristics to the composition which contribute to the stability of the composition. For example, the structuring agent tends to assist in the formation of the liquid crystalline gel network structures. The structuring agent may also function as an emulsifier or surfactant. Preferred compositions of this invention contain from about 0.1% to about 20%, more preferably from about 0.1% to about 10%, still more preferably from about 0.5% to about 9%, of one or more structuring agents.

Preferred structuring agents are those having an HLB of from about 1 to about 8 and having a melting point of at least about 450 C. Suitable structuring agents are those selected from saturated C₁₄ to C₃₀ fatty alcohols, saturated C₁₆ to C₃₀ fatty alcohols containing from about 1 to about 5 moles of ethylene oxide, saturated C₁₆ to C₃₀ diols, saturated C₁₆ to C₃₀ monoglycerol ethers, saturated C₁₆ to C₃₀ hydroxy fatty acids, C₁₄ to C₃₀ hydroxylated and nonhydroxylated saturated fatty acids, C₁₄ to C₃₀ saturated ethoxylated fatty acids, amines and alcohols containing from about 1 to about 5 moles of ethylene oxide diols, C₁₄ to C₃₀ saturated glyceryl mono esters with a monoglyceride content of at least 40%, C₁₄ to C₃₀ saturated polyglycerol esters having from about 1 to about 3 alkyl group and from about 2 to about 3 saturated glycerol units, C₁₄ to C₃₀ glyceryl mono ethers, C₁₄ to C₃₀ sorbitan mono/diesters, C₁₄ to C₃₀ saturated ethoxylated sorbitan mono/diesters with about 1 to about 5 moles of ethylene oxide, C₁₄ to C₃₀ saturated methyl glucoside esters, C₁₄ to C₃₀ saturated sucrose mono/diesters, C₁₄ to C₃₀ saturated ethoxylated methyl glucoside esters with about 1 to about 5 moles of ethylene oxide, C₁₄ to C₃₀ saturated polyglucosides having an average of between 1 to 2 glucose units and mixtures thereof, having a melting point of at least about 45° C.

The preferred structuring agents of the present invention are selected from stearic acid, palmitic acid, stearyl alcohol, cetyl alcohol, behenyl alcohol, stearic acid, palmitic acid, the polyethylene glycol ether of stearyl alcohol having an average of about 1 to about 5 ethylene oxide units, the polyethylene glycol ether of cetyl alcohol having an average of about 1 to about 5 ethylene oxide units, and mixtures thereof. More preferred structuring agents of the present invention are selected from stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof. Even more preferred structuring agents are selected from stearic acid, palmitic acid, stearyl alcohol, cetyl alcohol, behenyl alcohol, steareth-2, and mixtures thereof.

Thickening Agent (including thickeners and gelling agents)

The compositions of the present invention can contain one or more thickening agents, preferably from about 0.1% to about 5%, more preferably from about 0.1% to about 4%, and still more preferably from about 0.25% to about 3%, by weight of the composition.

Nonlimiting classes of thickening agents include those selected from the following:

Carboxylic Acid Polymers

These polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Polymers useful in the present invention are more fully described in U.S. Pat. No. 5,087,445, to Haffey et al., issued Feb. 11, 1992; U.S. Pat. No. 4,509,949, to Huang et al., issued Apr. 5, 1985; U.S. Pat. No. 2,798,053, to Brown, issued Jul. 2, 1957; and in CTFA International Cosmetic Ingredient Dictionary, Fourth Edition, 1991, pp. 12 and 80.

Examples of commercially available carboxylic acid polymers useful herein include the carbomers, which are homopolymers of acrylic acid crosslinked with allyl ethers of sucrose or pentaerytritol. The carbomers are available as the Carbopol® 900 series from B. F. Goodrich (e.g., Carbopol® 954). In addition, other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀-C₃₀ alkyl acrylate crosspolymers and are commercially available as Carbopol® 1342, Carbopol® 1382, Pemulen TR-1, and Pemulen TR 2, from B. F. Goodrich. In other words, examples of carboxylic acid polymer thickeners useful herein are those selected from carbomers, acrylates/C₁₀-C₃₀ alkyl acrylate crosspolymers, and mixtures thereof. Especially useful are combinations with the ingredient range called LUBRAJELS offered by UNITED GUARDIAN, some of them described in WO 97/47310 of Jun. 12, 1996.

b) Crosslinked Polyacrylate Polymers

The compositions of the present invention can optionally contain crosslinked polyacrylate polymers useful as thickeners or gelling agents including both cationic and nonionic polymers, with the cationics being generally preferred. Examples of useful crosslinked nonionic polyacrylate polymers and crosslinked cat-ionic polyacrylate polymers are those described in U.S. Pat. No. 5,100,660, to Hawe et al., issued Mar. 31, 1992; U.S. Pat. No. 4,849,484, to Heard, issued Jul. 18, 1989; U.S. Pat. No. 4,835,206, to Farrar et al., issued May 30, 1989; U.S. Pat. No. 4,628,078 to Glover et al. issued Dec. 9, 1986; U.S. Pat. No. 4,599,379 to Flesher et al. issued Jul. 8, 1986; and EP 228,868, to Farrar et al., published Jul. 15, 1987.

c) Polyacrylamide Polymers

The compositions of the present invention can optionally contain polyacrylamide polymers, especially nonionic polyacrylamide polymers including substituted branched or unbranched polymers. More preferred among these polyacrylamide polymers is the nonionic polymer given the CTFA designation polyacrylamide and isoparaffin and laureth-7, available under the Tradename Sepigel 305 from Seppic Corporation (Fairfield, N.J.).

Other polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS500W, SSSA100H, from Lipo Chemicals, Inc. (Paterson, N.J.).

d) Polysaccharides

A wide variety of polysaccharides are useful herein. “Polysaccharides” refer to gelling agents which contain a backbone of repeating sugar (i.e., carbohydrate) units. Nonlimiting examples of polysaccharide gelling agents include those selected from cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl substituted celluloses. In these polymers, the Irimeste groups of the cellulose polymer is hydroxyalkylated (preferably hydroxyethylated or hydroxypropylated) to form a hydroxyalkylated cellulose which is then further modified with a C₁₀-C₃₀ straight chain or branched chain alkyl group through an ether linkage. Typically these polymers are ethers of C₁₀-C₃₀ straight or branched chain alcohols with hydroxyalkyl-celluloses. Examples of alkyl groups useful herein include those selected from stearyl, isostearyl, lauryl, myristyl, cetyl, isocetyl, cocoyl (i.e. alkyl groups derived from the alcohols of coconut oil), palmityl, oleyl, linoleyl, linolenyl, ricinoleyl, behenyl, and mixtures thereof. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename Natrosol® CS Plus from Aqualon Corporation (Wilmington, Del.).

Other useful polysaccharides include scleroglucans which are a linear chain of (1-3) linked glucose units with a (1-6) linked glucose every three units, a commercially available example of which is Clearogel™ CS11 from Michel Mercier Products Inc. (Mountainside, N.J.).

e) Gums

Other thickening and gelling agents useful herein include materials which are primarily derived from natural sources. Nonlimiting examples of these gelling agent gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof.

Dermatologically-Acceptable Carrier

The compositions of the invention may be used in various cosmetic and/or personal care products, for example, hair care products, such as lotions, gels, sprays, and the like, hair sunscreen compositions, shampoos, hair conditioners, and hair coloring products. Therefore, in addition to any of the above cited skin care or hair care peptides and other actives, the cosmetic compositions described in the present invention may often include as an additional ingredient a dermatologically acceptable carrier. The form of the carrier and the final product resulting from the combination of the saccharose substitutes with any additional active and with the carrier may be any of the following: liquids, gels, creams, water-in-oil and oil-in-water, and silicone emulsions, foams; they may be clear or opaque; and may be formulated as both aqueous and non-aqueous preparations, including but not limited to topical preparations.

The nature of the dermatologically acceptable carrier, the nature of the final product, and the methods of preparing those need not be described here in detail; many examples can be found in the available literatures, such as PCT application No. WO 00/62743 filed by Larry R. Robinson et al. on Apr. 19, 2000, published on Oct. 26, 2000, or, more generally, in Milady's Standard Textbook of Cosmetology, (Delmar Learning 2000) or in Formulation Technology: Emulsions, Suspensions, Solid Forms by Hans Mollet, Arnold Grubenmann and Helen Payne (John Wiley & Sons, Jan. 23, 2001), or in Chemistry and Technology of the Cosmetics and Toiletries Industry by Clifford Williams Schmitt, (Kluwer Academic Publishers, July 1996), all hereby incorporated. Fiedler's Encyclopedia of Excipients, fifth edition (Editio Cantor Verlag 2002) is also a useful guide for the formulator skilled in the art of developing cosmetic carriers. All ingredients listed therein may in one way or another be combined to form a dermatologically acceptable carrier and/or used as an additional ingredient for the cosmetic compositions of the invention.

A safe and effective amount of carrier is from about 50% to about 99.99%, preferably from about 80% to about 99.9%, more preferably from about 90% to about 98%, and even more preferably from about 90% to about 95% of the composition.

The carrier can be in a wide variety of forms. For example, emulsion carriers, including, but not limited to, oil-in-water, water-in-oil, water-in-oil-in-water, and oil-in-water-in-silicone emulsions, are useful herein.

Preferred carriers contain an emulsion such as oil-in-water emulsions, water-in-oil emulsions, and water-in-silicone emulsions. As will be understood by the skilled artisan, a given component will distribute primarily into either the water or oil/silicone phase, depending on the water solubility/dispersibility of the component in the composition. Oil-in-water emulsions are especially preferred.

Emulsions according to the present invention generally contain a solution as described above and a lipid or oil. Lipids and oils may be derived from animals, plants, or petroleum and may be natural or synthetic (i.e., man-made). Preferred emulsions also contain a humectant, such as glycerin. Emulsions will preferably further contain from about 0.01% to about 10%, more preferably from about 0.1% to about 5%, of an emulsifier, based on the weight of the carrier. Emulsifiers may be nonionic, anionic or cationic. Suitable emulsifiers are disclosed in, for example, U.S. Pat. No. 3,755,560, issued Aug. 28, 1973, Dickert et al.; U.S. Pat. No. 4,421,769, issued Dec. 20, 1983, Dixon et al.; and McCutcheon's Detergents and Emulsifiers, North American Edition, pages 317-324 (1986).

The emulsion may also contain an anti-foaming agent to minimize foaming upon application to the keratinous tissue. Anti-foaming agents include high molecular weight silicones and other materials well known in the art for such use.

Suitable emulsions may have a wide range of viscosities, depending on the desired product form. Exemplary low viscosity emulsions, which are preferred, have a viscosity of about 50 centistokes or less, more preferably about 10 centistokes or less, still more preferably about 5 centistokes or less.

Water-in-silicone and oil-in-water emulsions are described in greater detail below.

Water-In-silicone Emulsion

Water-in-silicone emulsions contain a continuous silicone phase and a dispersed aqueous phase.

Continuous Silicone Phase

Preferred water-in-silicone emulsions of the present invention contain from about 1% to about 60%, preferably from about 5% to about 40%, more preferably from about 10% to about 20%, by weight of a continuous silicone phase. The continuous silicone phase exists as an external phase that contains or surrounds the discontinuous aqueous phase described hereinafter.

The continuous silicone phase contains a polyorganosiloxane oil. A preferred water-in-silicone emulsion system is formulated to provide an oxidatively stable vehicle for the retinoid. The continuous silicone phase of these preferred emulsions contain between about 50% and about 99.9% by weight of organopolysiloxane oil and less than about 50% by weight of a non-silicone oil. In an especially preferred embodiment, the continuous silicone phase contains at least about 50%, preferably from about 60% to about 99.9%, more preferably from about 70% to about 99.9%, and even more preferably from about 80% to about 99.9%, polyorganosiloxane oil by weight of the continuous silicone phase, and up to about 50% non-silicone oils, preferably less about 40%, more preferably less than about 30%, even more preferably less than about 10%, and even more preferably less than about 2%, by weight of the continuous silicone phase. These preferred emulsion systems provide more oxidative stability to the retinoid over extended periods of time than comparable water-in-oil emulsions containing lower concentrations of the polyorganosiloxane oil. Concentrations of non-silicone oils in the continuous silicone phase are minimized or avoided altogether so as to further enhance oxidative stability of the selected retinoid in the compositions. Water-in-silicone emulsions of this type are described in PCT Application WO 97/21423, published Jun. 19, 1997.

The organopolysiloxane oil for use in the composition may be volatile, non-volatile, or a mixture of volatile and non-volatile silicones. The term “nonvolatile” as used in this context refers to those silicones that are liquid under ambient conditions and have a flash point (under one atmospheric of pressure) of or greater than about 100° C. The term “volatile” as used in this context refers to all other silicone oils. Suitable organopolysiloxanes can be selected from a wide variety of silicones spanning a broad range of volatilities and viscosities. Examples of suitable organopolysiloxane oils include polyalkylsiloxanes, cyclic polyalkylsiloxanes, and polyalkylarylsiloxanes.

Polyalkylsiloxanes useful in the composition herein include polyalkylsiloxanes with viscosities of from about 0.5 to about 1,000,000 centistokes at 25° C. Such polyalkylsiloxanes can be represented by the general chemical formula R₃SiO[R₂SiO]ₓSiR₃ wherein R is an alkyl group having from one to about 30 carbon atoms (preferably R is methyl or ethyl, more preferably methyl; also mixed alkyl groups can be used in the same molecule), and x is an integer from 0 to about 10,000, chosen to achieve the desired molecular weight which can range to over about 10,000,000. Commercially available polyalkylsiloxanes include the polydimethylsiloxanes, which are also known as dimethicones, examples of which include the Vicasil® series sold by General Electric Company and the Dow Corning® 200 series sold by Dow Corning Corporation. Specific examples of suitable polydimethylsiloxanes include Dow Corning® 200 fluid having a viscosity of 0.65 centistokes and a boiling point of 100° C., Dow Corning® 225 fluid having a viscosity of 10 centistokes and a boiling point greater than 200° C., and Dow Corning® 200 fluids having viscosities of 50, 350, and 12,500 centistokes, respectively, and boiling points greater than 200° C. Suitable dimethicones include those represented by the chemical formula (CH₃)₃SiO[(CH₃)₂SiO]ₓ[CH₃RsiO]_{y}Si(CH₃)₃ wherein R is straight or branched chain alkyl having from two to about 30 carbon atoms and x and y are each integers of 1 or greater selected to achieve the desired molecular weight which can range to over about 10,000,000 amu. Examples of these alkyl-substituted dimethicones include cetyl dimethicone and lauryl dimethicone.

Cyclic polyalkylsiloxanes suitable for use in the composition include those represented by the chemical formula (SiR₂—O]ₙ wherein R is an alkyl group (preferably R is methyl or ethyl, more preferably methyl) and n is an integer from about 3 to about 8, more preferably n is an integer from about 3 to about 7, and still more preferably n is an integer from about 4 to about 6. When R is methyl, these materials are typically referred to as cyclomethicones. Commercially available cyclomethicones include Dow Corning® 244 fluid having a viscosity of 2.5 centistokes, and a boiling point of 172° C., which primarily contains the cyclomethicone tetramer (i.e. n=4), Dow Corning® 344 fluid having a viscosity of 2.5 centistokes and a boiling point of 178° C., which primarily contains the cyclomethicone pentamer (i.e. n=5), Dow Corning® 245 fluid having a viscosity of 4.2 centistokes and a boiling point of 205° C., which primarily contains a mixture of the cyclomethicone tetramer and pentamer (i.e. n=4 and 5), and Dow Corning® 345 fluid having a viscosity of 4.5 centistokes and a boiling point of 217°, which primarily contains a mixture of the cyclomethicone tetramer, pentamer, and hexamer (i.e. n=4, 5, and 6).

Also useful are materials such as trimethylsiloxysilicate, which is a polymeric material corresponding to the general chemical formula [(CH₂)₃SiO_{1/2}]ₓ[SiO₂]_{y}, wherein x is an integer from about 1 to about 500 and y is an integer from about 1 to about 500. A commercially available trimethylsiloxysilicate is sold as a mixture with dimethicone as Dow Corning® 593 fluid.

Dimethiconols are also suitable for use in the composition. These compounds can be represented by the chemical formulas R₃SiO[R₂SiO]ₓSiR₂OH and HOR₂SiO[R₂SiO]ₓSiR₂OH wherein R is an alkyl group (preferably R is methyl or ethyl, more preferably methyl) and x is an integer from 0 to about 500, chosen to achieve the desired molecular weight. Commercially available dimethiconols are typically sold as mixtures with dimethicone or cyclomethicone (e.g. Dow Corning® 1401, 1402, and 1403 fluids).

Polyalkylaryl siloxanes are also suitable for use in the composition. Polymethylphenyl siloxanes having viscosities from about 15 to about 65 centistokes at 25° C. are especially useful.

Preferred for use herein are organopolysiloxanes selected from polyalkylsiloxanes, alkyl substituted dimethicones, cyclomethicones, trimethylsiloxysilicates, dimethiconols, polyalkylaryl siloxanes, and mixtures thereof. More preferred for use herein are polyalkylsiloxanes and cyclomethicones. Preferred among the polyalkylsiloxanes are dimethicones.

As stated above, the continuous silicone phase may contain one or more non-silicone oils. Concentrations of non-silicone oils in the continuous silicone phase are preferably minimized or avoided altogether so as to further enhance oxidative stability of the selected retinoid in the compositions. Suitable non-silicone oils have a melting point of about 25° C. or less under about one atmosphere of pressure. Examples of non-silicone oils suitable for use in the continuous silicone phase are those well known in the chemical arts in topical personal care products in the form of water-in-oil emulsions, e.g., mineral oil, vegetable oils, synthetic oils, semisynthetic oils, etc.

(2) Dispersed Aqueous Phase

The topical compositions of the present invention contain from about 30% to about 90%, more preferably from about 50% to about 85%, and still more preferably from about 70% to about 80% of a dispersed aqueous phase. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase. The dispersed aqueous phase is a dispersion of small aqueous particles or droplets suspended in and surrounded by the continuous silicone phase described hereinbefore.

The aqueous phase can be water, or a combination of water and one or more water soluble or dispersible ingredients. Nonlimiting examples of such ingredients include thickeners, acids, bases, salts, chelants, gums, water-soluble or dispersible alcohols and polyols, buffers, preservatives, sunscreening agents, colorings, and the like.

The topical compositions of the present invention will typically contain from about 25% to about 90%, preferably from about 40% to about 80%, more preferably from about 60% to about 80%, water in the dispersed aqueous phase by weight of the composition.

(3) Emulsifier for Dispersing the Aqueous Phase

The water-in-silicone emulsions of the present invention preferably contain an emulsifier. In a preferred embodiment, the composition contains from about 0.1% to about 10% emulsifier, more preferably from about 0.5% to about 7.5%, still more preferably from about 1% to about 5%, emulsifier by weight of the composition. The emulsifier helps disperse and suspend the aqueous phase within the continuous silicone phase.

A wide variety of emulsifying agents can be employed herein to form the preferred water-in-silicone emulsion. Known or conventional emulsifying agents can be used in the composition, provided that the selected emulsifying agent is chemically and physically compatible with components of the composition of the present invention, and provides the desired dispersion characteristics. Suitable emulsifiers include silicone emulsifiers, non-silicon-containing emulsifiers, and mixtures thereof, known by those skilled in the art for use in topical personal care products. Preferably these emulsifiers have an HLB value of or less than about 14, more preferably from about 2 to about 14, and still more preferably from about 4 to about 14. Emulsifiers having an HLB value outside of these ranges can be used in combination with other emulsifiers to achieve an effective weighted average HLB for the combination that falls within these ranges.

Silicone emulsifiers are preferred. A wide variety of silicone emulsifiers are useful herein. These silicone emulsifiers are typically organically modified organopolysiloxanes, also known to those skilled in the art as silicone surfactants. Useful silicone emulsifiers include dimethicone copolyols. These materials are polydimethyl siloxanes which have been modified to include polyether side chains such as polyethylene oxide chains, polypropylene oxide chains, mixtures of these chains, and polyether chains containing moieties derived from both ethylene oxide and propylene oxide. Other examples include alkyl-modified dimethicone copolyols, i.e., compounds which contain C₂-C₃₀ pendant side chains. Still other useful dimethicone copolyols include materials having various cationic, anionic, amphoteric, and zwitterionic pendant moieties.

The dimethicone copolyol emulsifiers useful herein can be described by the following general structure: 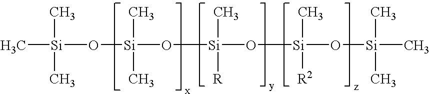
wherein R is C₁-C₃₀ straight, branched, or cyclic alkyl and R² is selected from the group consisting of 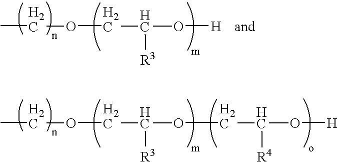
wherein n is an integer from 3 to about 10; R³ and R⁴ are selected from the group consisting of H and C1-C6 straight or branched chain alkyl such that R³ and R⁴ are not simultaneously the same; and m, o, x, and y are selected such that the molecule has an overall molecular weight from about 200 to about 10,000,000 amu, with m, o, x, and y being independently selected from integers of zero or greater such that m and o are not both simultaneously zero, and z being independently selected from integers of 1 or greater. It is recognized that positional isomers of these copolyols can be achieved. The chemical representations depicted above for the R² moieties containing the R³ and R⁴ groups are not meant to be limiting but are shown as such for convenience.

Also useful herein, although not strictly classified as dimethicone copolyols, are silicone surfactants as depicted in the structures in the previous paragraph wherein R² is: —(CH₂)_{n—O—R}⁵ wherein R⁵ is a cationic, anionic, amphoteric, or zwitterionic moiety.

Nonlimiting examples of dimethicone copolyols and other silicone surfactants useful as emulsifiers herein include polydimethylsiloxane polyether copolymers with pendant polyethylene oxide sidechains, polydimethylsiloxane polyether copolymers with pendant polypropylene oxide sidechains, polydimethylsiloxane polyether copolymers with pendant mixed polyethylene oxide and polypropylene oxide sidechains, polydimethylsiloxane polyether copolymers with pendant mixed poly(ethylene)(propylene)oxide sidechains, polydimethylsiloxane polyether copolymers with pendant organobetaine sidechains, polydimethylsiloxane polyether copolymers with pendant carboxylate sidechains, polydimethylsiloxane polyether copolymers with pendant quaternary ammonium sidechains; and also further modifications of the preceding copolymers containing pendant C₂-C₃₀ straight, branched, or cyclic alkyl moieties. Examples of commercially available dimethicone copolyols useful herein sold by Dow Corning Corporation are Dow Corning® 190, 193, Q2-5220, 2501 Wax, 2-5324 fluid, and 3225C (this later material being sold as a mixture with cyclomethicone). Cetyl dimethicone copolyol is commercially available as a mixture with polyglyceryl-4 isostearate (and) hexyl laurate and is sold under the tradename ABIL® WE-09 (available from Goldschmidt). Cetyl dimethicone copolyol is also commercially available as a mixture with hexyl laurate (and) polyglyceryl-3 oleate (and) cetyl dimethicone and is sold under the tradename ABIL® WS-08 (also available from Goldschmidt). Other nonlimiting examples of dimethicone copolyols also include lauryl dimethicone copolyol, dimethicone copolyol acetate, diemethicone copolyol adipate, dimethicone copolyolamine, dimethicone copolyol behenate, dimethicone copolyol butyl ether, dimethicone copolyol Irimeste stearate, dimethicone copolyol isostearate, dimethicone copolyol laurate, dimethicone copolyol methyl ether, dimethicone copolyol phosphate, and dimethicone copolyol stearate. See International Cosmetic Ingredient Dictionary, Fifth Edition, 1993.

Dimethicone copolyol emulsifiers useful herein are described, for example, in U.S. Pat. No. 4,960,764, to Figueroa, Jr. et al., issued Oct. 2, 1990; European Patent No. EP 330,369, to SanoGueira, published Aug. 30, 1989; G. H. Dahms, et al., “New Formulation Possibilities Offered by Silicone Copolyols,” Cosmetics & Toiletries, vol. 110, pp. 91-100, March 1995; M. E. Carlotti et al., “Optimization of W/O—S Emulsions And Study Of The Quantitative Relationships Between Ester Structure And Emulsion Properties,” J. Dispersion Science And Technology, 13(3), 315-336 (1992); P. Hameyer, “Comparative Technological Investigations of Organic and Organosilicone Emulsifiers in Cosmetic Water-in-Oil Emulsion Preparations,” HAPPI 28(4), pp. 88-128 (1991); J. Smid-Korbar et al., “Efficiency and usability of silicone surfactants in emulsions,” Provisional Communication International Journal of Cosmetic Science, 12, 135-139 (1990); and D. G. Krzysik et al., “A New Silicone Emulsifier For Water-in-Oil Systems,” Drug and Cosmetic Industry, vol. 146(4) pp. 28-81 (April 1990).

Among the non-silicone-containing emulsifiers useful herein are various non-ionic and anionic emulsifying agents such as sugar esters and polyesters, alkoxylated sugar esters and polyesters, C₁-C₃₀ fatty acid esters of C₁-C₃₀ fatty alcohols, alkoxylated derivatives of C₁-C₃₀ fatty acid esters of C₁-C₃₀ fatty alcohols, alkoxylated ethers of C₁-C₃₀ fatty alcohols, polyglyceryl esters of C₁-C₃₀ fatty acids, C₁-C₃₀ esters of polyols, C₁-C₃₀ ethers of polyols, alkyl phosphates, polyoxyalkylene fatty ether phosphates, fatty acid amides, acyl lactylates, soaps, and mixtures thereof. Other suitable emulsifiers are described, for example, in McCutcheon's, Detergents and Emulsifiers, North American Edition (1986), published by Allured Publishing Corporation; U.S. Pat. No. 5,011,681 to Ciotti et al., issued Apr. 30, 1991; U.S. Pat. No. 4,421,769 to Dixon et al., issued Dec. 20, 1983; and U.S. Pat. No. 3,755,560 to Dickert et al., issued Aug. 28, 1973.

Nonlimiting examples of these non-silicon-containing emulsifiers include: polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, PEG-100 stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, steareth-20, ceteareth-20, PPG-2 methyl glucose ether distearate, ceteth-10, diethanolamine cetyl phosphate, glyceryl stearate, PEG-100 stearate, and mixtures thereof.

Oil-In-Water Emulsions

Other preferred topical carriers include oil-in-water emulsions, having a continuous aqueous phase and a hydrophobic, water-insoluble phase (“oil phase”) dispersed therein. Examples of suitable oil-in-water emulsion carriers are described in U.S. Pat. No. 5,073,371, to Turner, D. J. et al., issued Dec. 17, 1991, and U.S. Pat. No. 5,073,372, to Turner, D. J. et al., issued Dec. 17, 1991. An especially preferred oil-in-water emulsion, containing a structuring agent, hydrophilic surfactant and water, is described in detail hereinafter.

Structuring Agent

A preferred oil-in-water emulsion contains a structuring agent to assist in the formation of a liquid crystalline gel network structure. Without being limited by theory, it is believed that the structuring agent assists in providing rheological characteristics to the composition which contribute to the stability of the composition. The structuring agent may also function as an emulsifier or surfactant. Preferred compositions of this invention contain from about 0.5% to about 20%, more preferably from about 1% to about 10%, even more preferably from about 1% to about 5%, by weight of the composition, of a structuring agent.

Structuring agents of the present invention can include stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof. More preferred structuring agents of the present invention are selected from stearyl alcohol, cetyl alcohol, behenyl alcohol, the polyethylene glycol ether of stearyl alcohol having an average of about 2 ethylene oxide units (steareth-2), the polyethylene glycol ether of stearyl alcohol having an average of about 21 ethylene oxide units (steareth-21), the polyethylene glycol ether of cetyl alcohol having an average of about 2 ethylene oxide units, and mixtures thereof. Even more preferred structuring agents are selected from stearic acid, palmitic acid, stearyl alcohol, cetyl alcohol, behenyl alcohol, steareth-2, steareth-21, and mixtures thereof.

Hydrophilic Surfactant

The compositions may also contain at least one hydrophilic surfactant. The surfactant, at a minimum, must be hydrophilic enough to disperse in water.

Preferred hydrophilic surfactants are selected from nonionic surfactants. Among the nonionic surfactants that are useful herein are those that can be broadly defined as condensation products of long chain alcohols, e.g. C₈₋₃₀ alcohols, with sugar or starch polymers, i.e., glycosides. These compounds can be represented by the formula (S)ₙ—O—R wherein S is a sugar moiety such as glucose, fructose, mannose, and galactose; n is an integer of from about 1 to about 1000, and R is a C₈₋₃₀ alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants include those wherein S is a glucose moiety, R is a C₈₋₂₀ alkyl group, and n is an integer of from about 1 to about 9. Commercially available examples of these surfactants include decyl polyglucoside (available as APG 325 CS from Henkel) and lauryl polyglucoside (available as APG 600 CS and 625 CS from Henkel).

Other useful nonionic surfactants include the condensation products of alkylene oxides with fatty acids (i.e. alkylene oxide esters of fatty acids). These materials have the general formula RCO(X)ₙOH wherein R is a C₁₀₋₃₀ alkyl group, X is —OCH₂CH₂— (i.e. derived from ethylene glycol or oxide) or —OCH₂CH₃— (i.e. derived from propylene glycol or oxide), and n is an integer from about 6 to about 200. Other nonionic surfactants are the condensation products of alkylene oxides with 2 moles of fatty acids (i.e. alkylene oxide diesters of fatty acids). These materials have the general formula RCO(X)ₙOOCR wherein R is a C₁₀₋₃₀ alkyl group, X is —OCH₂CH₂— (i.e. derived from ethylene glycol or oxide) or —OCH₂CH₃— (i.e. derived from propylene glycol or oxide), and n is an integer from about 6 to about 100. Other nonionic surfactants are the condensation products of alkylene oxides with fatty alcohols (i.e. alkylene oxide ethers of fatty alcohols). These materials have the general formula R(X)ₙOR′ wherein R is a C₁₀₋₃₀ alkyl group, X is —OCH₂CH₂— (i.e., derived from ethylene glycol or oxide) or —OCH₂CH₃— (i.e., derived from propylene glycol or oxide), n is an integer from about 6 to about 100, and R′ is H or a C₁₀₋₃₀ alkyl group. Still other nonionic surfactants are the condensation products of alkylene oxides with both fatty acids and fatty alcohols [i.e. wherein the polyalkylene oxide portion is esterified on one end with a fatty acid and etherified (i.e. connected via an ether linkage) on the other end with a fatty alcohol]. These materials have the general formula RCO(X)ₙOR′ wherein R and R′ are C₁₀₋₃₀ alkyl groups, X is —OCH₂CH₂— (i.e., derived from ethylene glycol or oxide) or —OCH₂CH₃— (derived from propylene glycol or oxide), and n is an integer from about 6 to about 100. Nonlimiting examples of these alkylene oxide derived nonionic surfactants include ceteth-6, ceteth-10, ceteth-12, ceteareth-6, ceteareth-10, ceteareth-12, steareth-6, steareth-10, steareth-12, steareth-21, PEG-6 stearate, PEG-10 stearate, PEG-100 stearate, PEG-12 stearate, PEG-20 glyceryl stearate, PEG-80 glyceryl tallowate, PEG-10 glyceryl stearate, PEG-30 glyceryl cocoate, PEG-80 glyceryl cocoate, PEG-200 glyceryl tallowate, PEG-8 dilaurate, PEG-10 distearate, and mixtures thereof.

Still other useful nonionic surfactants include polyhydroxy fatty acid amide surfactants corresponding to the structural formula: 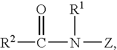
wherein: R¹ is H, C₁-C₄ alkyl, 2-hydroxyethyl, 2-hydroxy-propyl, preferably C₁-C₄ alkyl, more preferably methyl or ethyl, most preferably methyl; R² is C₅-C₃, alkyl or alkenyl, preferably C₇-C₁₉ alkyl or alkenyl, more preferably C₉-C₁₇ alkyl or alkenyl, most preferably C₁₁-C₁₅ alkyl or alkenyl; and Z is a polhydroxyhydrocarbyl moiety having a linear hydrocarbyl chain with a least 3 hydroxyls directly connected to the chain, or an alkoxylated derivative (preferably ethoxylated or propoxylated) thereof. Z preferably is a sugar moiety selected from the group consisting of glucose, fructose, maltose, lactose, galactose, mannose, xylose, and mixtures thereof. An especially preferred surfactant corresponding to the above structure is coconut alkyl N-methyl glucoside amide (i.e., wherein the R²CO— moiety is derived from coconut oil fatty acids). Processes for making compositions containing polyhydroxy fatty acid amides are disclosed, for example, in G.B. Patent Specification 809,060, published Feb. 18, 1959, by Thomas Hedley & Co., Ltd.; U.S. Pat. No. 2,965,576, to E. R. Wilson, issued Dec. 20, 1960; U.S. Pat. No. 2,703,798, to A. M. Schwartz, issued Mar. 8, 1955; and U.S. Pat. No. 1,985,424, to Piggott, issued Dec. 25, 1934; which are incorporated herein by reference in their entirety.

Preferred among the nonionic surfactants are those selected from the group consisting of steareth-21, ceteareth-20, ceteareth-12, sucrose cocoate, steareth-100, PEG-100 stearate, and mixtures thereof.

Other nonionic surfactants suitable for use herein include sugar esters and polyesters, alkoxylated sugar esters and polyesters, C₁-C₃₀ fatty acid esters of C₁-C₃₀ fatty alcohols, alkoxylated derivatives of C₁-C₃₀ fatty acid esters of C₁-C₃₀ fatty alcohols, alkoxylated ethers of C₁-C₃₀ fatty alcohols, polyglyceryl esters of C₁-C₃₀ fatty acids, C₁-C₃₀ esters of polyols, C₁-C₃₀ ethers of polyols, alkyl phosphates, polyoxyalkylene fatty ether phosphates, fatty acid amides, acyl lactylates, and mixtures thereof. Nonlimiting examples of these emulsifiers include: polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, PPG-2 methyl glucose ether distearate, PEG-100 stearate, and mixtures thereof.

Another group of non-ionic surfactants useful herein are fatty acid ester blends based on a mixture of sorbitan or sorbitol fatty acid ester and sucrose fatty acid ester, the fatty acid in each instance being preferably C₈-C₂₄, more preferably C₁₀-C₂₀. The preferred fatty acid ester emulsifier is a blend of sorbitan or sorbitol C₁₆-C₂₀ fatty acid ester with sucrose C₁₀-C₁₆ fatty acid ester, especially sorbitan stearate and sucrose cocoate. This is commercially available from ICI under the trade name Arlatone 2121.

Other suitable surfactants useful herein include a wide variety of cationic, anionic, zwitterionic, and amphoteric surfactants such as are known in the art and discussed more fully below. See, e.g., McCutcheon's, Detergents and Emulsifiers, North American Edition (1986), published by Allured Publishing Corporation; U.S. Pat. No. 5,011,681 to Ciotti et al., issued Apr. 30, 1991; U.S. Pat. No. 4,421,769 to Dixon et al., issued Dec. 20, 1983; and U.S. Pat. No. 3,755,560 to Dickert et al., issued Aug. 28, 1973; these four references are incorporated herein by reference in their entirety. The hydrophilic surfactants useful herein can contain a single surfactant, or any combination of suitable surfactants. The exact surfactant (or surfactants) chosen will depend upon the pH of the composition and the other components present.

Also useful herein are cationic surfactants, especially dialkyl quaternary ammonium compounds or “quats”, examples of which are described in U.S. Pat. No. 5,151,209; U.S. Pat. No. 5,151,210; U.S. Pat. No. 5,120,532; U.S. Pat. No. 4,387,090; U.S. Pat. No. 3,155,591; U.S. Pat. No. 3,929,678; U.S. Pat. No. 3,959,461; McCutcheon's Detergents & Emulsifiers, (North American edition 1979) M. C. Publishing Co.; and Schwartz, et al., Surface Active Agents, Their Chemistry and Technology, New York: Interscience Publishers, 1949; which descriptions are incorporated herein by reference. The cationic surfactants useful herein include cationic ammonium salts such as those having the formula: 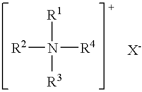
wherein R¹, is an alkyl group having from about 12 to about 30 carbon atoms, or an aromatic, aryl or alkaryl group having from about 12 to about 30 carbon atoms; R², R³, and R⁴ are independently selected from hydrogen, an alkyl group having from about 1 to about 22 carbon atoms, or aromatic, aryl or alkaryl groups having from about 12 to about 22 carbon atoms; and X is any compatible anion, preferably selected from chloride, bromide, iodide, acetate, phosphate, nitrate, sulfate, methyl sulfate, ethyl sulfate, tosylate, lactate, citrate, glycolate, and mixtures thereof. Additionally, the alkyl groups of R¹, R², R³, and R⁴ can also contain ester and/or ether linkages, or irimeste or amino group substituents (e.g., the alkyl groups can contain polyethylene glycol and polypropylene glycol moieties).

More preferably, R¹ is an alkyl group having from about 12 to about 22 carbon atoms; R² is selected from H or an alkyl group having from about 1 to about 22 carbon atoms; R³ and R⁴ are independently selected from H or an alkyl group having from about 1 to about 3 carbon atoms; and X is as described previously.

Still more preferably, R¹ is an alkyl group having from about 12 to about 22 carbon atoms; R², R³, and R⁴ are selected from H or an alkyl group having from about 1 to about 3 carbon atoms; and X is as described previously.

Alternatively, other useful cationic emulsifiers include amino-amides, wherein in the above structure R¹ is alternatively R⁵CONH— (CH₂)ₙ, wherein R⁵ is an alkyl group having from about 12 to about 22 carbon atoms, and n is an integer from about 2 to about 6, more preferably from about 2 to about 4, and still more preferably from about 2 to about 3. Nonlimiting examples of these cationic emulsifiers include stearamidopropyl PG-dimonium chloride phosphate, behenamidopropyl PG dimonium chloride, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, and mixtures thereof. Especially preferred is behenamidopropyl PG dimonium chloride.

Nonlimiting examples of quaternary ammonium salt cationic surfactants include those selected from cetyl ammonium chloride, cetyl ammonium bromide, lauryl ammonium chloride, lauryl ammonium bromide, stearyl ammonium chloride, stearyl ammonium bromide, cetyl dimethyl ammonium chloride, cetyl dimethyl ammonium bromide, lauryl dimethyl ammonium chloride, lauryl dimethyl ammonium bromide, stearyl dimethyl ammonium chloride, stearyl dimethyl ammonium bromide, cetyl trimethyl ammonium chloride, cetyl trimethyl ammonium bromide, lauryl trimethyl ammonium chloride, lauryl trimethyl ammonium bromide, stearyl trimethyl ammonium chloride, stearyl trimethyl ammonium bromide, lauryl dimethyl ammonium chloride, stearyl dimethyl cetyl ditallow dimethyl ammonium chloride, dicetyl ammonium chloride, dicetyl ammonium bromide, dilauryl ammonium chloride, dilauryl ammonium bromide, distearyl ammonium chloride, distearyl ammonium bromide, dicetyl methyl ammonium chloride, dicetyl methyl ammonium bromide, dilauryl methyl ammonium chloride, dilauryl methyl ammonium bromide, distearyl methyl ammonium chloride, distearyl methyl ammonium bromide, and mixtures thereof. Additional quaternary ammonium salts include those wherein the C₁₂ to C₃₀ alkyl carbon chain is derived from a tallow fatty acid or from a coconut fatty acid. The term “tallow” refers to an alkyl group derived from tallow fatty acids (usually hydrogenated tallow fatty acids), which generally have mixtures of alkyl chains in the C₁₆ to C₁₈ range. The term “coconut” refers to an alkyl group derived from a coconut fatty acid, which generally have mixtures of alkyl chains in the C₁₂ to C₁₄ range. Examples of quaternary ammonium salts derived from these tallow and coconut sources include ditallow dimethyl ammonium chloride, ditallow dimethyl ammonium methyl sulfate, di(hydrogenated tallow)dimethyl ammonium chloride, di(hydrogenated tallow)dimethyl ammonium acetate, ditallow dipropyl ammonium phosphate, ditallow dimethyl ammonium nitrate, di(coconutalkyl)dimethyl ammonium chloride, di(coconutalkyl)dimethyl ammonium bromide, tallow ammonium chloride, coconut ammonium chloride, stearamidopropyl PG-dimonium chloride phosphate, stearamidopropyl ethyldimonium ethosulfate, stearamidopropyl dimethyl(myristyl acetate)ammonium chloride, stearamidopropyl dimethyl cetearyl ammonium tosylate, stearamidopropyl dimethyl ammonium chloride, stearamidopropyl dimethyl ammonium lactate, and mixtures thereof. An example of a quaternary ammonium compound having an alkyl group with an ester linkage is ditallowyl oxyethyl dimethyl ammonium chloride.

More preferred cationic surfactants are those selected from behenamidopropyl PG dimonium chloride, dilauryl dimethyl ammonium chloride, distearyl dimethyl ammonium chloride, dimyristyl dimethyl ammonium chloride, dipalmityl dimethyl ammonium chloride, distearyl dimethyl ammonium chloride, stearamidopropyl PG-dimonium chloride phosphate, stearamidopropyl ethyldiammonium ethosulfate, stearamidopropyl dimethyl(myristyl acetate)ammonium chloride, stearamidopropyl dimethyl cetearyl ammonium tosylate, stearamidopropyl dimethyl ammonium chloride, stearamidopropyl dimethyl ammonium lactate, and mixtures thereof.

Still more preferred cationic surfactants are those selected from behenamidopropyl PG dimonium chloride, dilauryl dimethyl ammonium chloride, distearyl dimethyl ammonium chloride, dimyristyl dimethyl ammonium chloride, dipalmityl dimethyl ammonium chloride, and mixtures thereof.

A preferred combination of cationic surfactant and structuring agent is behenamidopropyl PG dimonium chloride and/or behenyl alcohol, wherein the ratio is preferably optimized to maintained to enhance physical and chemical stability, especially when such a combination contains ionic and/or highly polar solvents. This combination is especially useful for delivery of sunscreening agents such as zinc oxide and octyl methoxycinnamate.

A wide variety of anionic surfactants are also useful herein. See, e.g., U.S. Pat. No. 3,929,678, to Laughlin et al., issued Dec. 30, 1975, which is incorporated herein by reference in its entirety. Nonlimiting examples of anionic surfactants include the alkoyl isethionates, and the alkyl and alkyl ether sulfates. The alkoyl isethionates typically have the formula RCO—OCH₂CH₂SO₃M wherein R is alkyl or alkenyl of from about 10 to about 30 carbon atoms, and M is a water-soluble cation such as ammonium, sodium, potassium and triethanolamine. Nonlimiting examples of these isethionates include those alkoyl isethionates selected from ammonium cocoyl isethionate, sodium cocoyl isethionate, sodium lauroyl isethionate, sodium stearoyl isethionate, and mixtures thereof.

The alkyl and alkyl ether sulfates typically have the respective formulae ROSO₃M and RO(C₂H₄O)ₓSO₃M, wherein R is alkyl or alkenyl of from about 10 to about 30 carbon atoms, x is from about 1 to about 10, and M is a water-soluble cation such as ammonium, sodium, potassium and triethanolamine. Another suitable class of anionic surfactants are the water-soluble salts of the organic, sulfuric acid reaction products of the general formula: R₁—SO₃-M, wherein R₁ is chosen from the group including a straight or branched chain, saturated aliphatic hydrocarbon radical having from about 8 to about 24, preferably about 10 to about 16, carbon atoms; and M is a cation. Still other anionic synthetic surfactants include the class designated as succinamates, olefin sulfonates having about 12 to about 24 carbon atoms, and β-alkyloxy alkane sulfonates. Examples of these materials are sodium lauryl sulfate and ammonium lauryl sulfate.

Other anionic materials useful herein are soaps (i.e., alkali metal salts, e.g., sodium or potassium salts) of fatty acids, typically having from about 8 to about 24 carbon atoms, preferably from about 10 to about 20 carbon atoms. The fatty acids used in making the soaps can be obtained from natural sources such as, for instance, plant or animal-derived glycerides (e.g., palm oil, coconut oil, soybean oil, castor oil, tallow, lard, etc.) The fatty acids can also be synthetically prepared. Soaps are described in more detail in U.S. Pat. No. 4,557,853.

Amphoteric and zwitterionic surfactants are also useful herein. Examples of amphoteric and zwitterionic surfactants which can be used in the compositions of the present invention are those which are broadly described as derivatives of aliphatic secondary and tertiary amines in which the aliphatic radical can be straight or branched chain and wherein one of the aliphatic substituents contains from about 8 to about 22 carbon atoms (preferably C₈-C₁₈) and one contains an anionic water solubilizing group, e.g., carboxy, sulfonate, sulfate, phosphate, or phosphonate. Examples are alkyl imino acetates, and iminodialkanoates and aminoalkanoates of the formulas RN[CH₂)ₘCO₂M]₂ and RNH(CH₂)ₘCO₂M wherein m is from 1 to 4, R is a C₈-C₂₂ alkyl or alkenyl, and M is H, alkali metal, alkaline earth metal ammonium, or alkanolammonium. Also included are imidazolinium and ammonium derivatives. Specific examples of suitable amphoteric surfactants include sodium 3-dodecyl-aminopropionate, sodium 3-dodecylaminopropane sulfonate, N-alkyltaurines such as the one prepared by reacting dodecylamine with sodium isethionate according to the teaching of U.S. Pat. No. 2,658,072 which is incorporated herein by reference in its entirety; N-higher alkyl aspartic acids such as those produced according to the teaching of U.S. Pat. No. 2,438,091 which is incorporated herein by reference in its entirety; and the products sold under the trade name “Miranol” and described in U.S. Pat. No. 2,528,378, which is incorporated herein by reference in its entirety. Other examples of useful amphoterics include phosphates, such as coamidopropyl PG-dimonium chloride phosphate (commercially available as Monaquat PTC, from Mona Corp.).

Other amphoteric or zwitterionic surfactants useful herein include betaines. Examples of betaines include the higher alkyl betaines, such as coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine (available as Lonzaine 16SP from Lonza Corp.), lauryl bis-(2-hydroxyethyl)carboxymethyl betaine, stearyl bis-(2-hydroxypropyl)carboxymethyl betaine, oleyl dimethyl gamma-carboxypropyl betaine, lauryl bis-(2-hydroxypropyl)alpha-carboxyethyl betaine, coco dimethyl sulfopropyl betaine, stearyl dimethyl sulfopropyl betaine, lauryl dimethyl sulfoethyl betaine, lauryl bis-(2-hydroxyethyl)sulfopropyl betaine, and amidobetaines and amidosulfobetaines (wherein the RCONH(CH₂)₃ radical is attached to the nitrogen atom of the betaine), oleyl betaine (available as amphoteric Velvetex OLB-50 from Henkel), and cocamidopropyl betaine (available as Velvetex BK-35 and BA-35 from Henkel).

Other useful amphoteric and zwitterionic surfactants include the sultaines and hydroxysultaines such as cocamidopropyl hydroxysultaine (available as Mirataine CBS from Rhone-Poulenc), and the alkanoyl sarcosinates corresponding to the formula RCON(CH₃)CH₂CH₂CO₂M wherein R is alkyl or alkenyl of about 10 to about 20 carbon atoms, and M is a water-soluble cation such as ammonium, sodium, potassium and trialkanolamine (e.g., triethanolamine), a preferred example of which is sodium lauroyl sarcosinate.

When the surfactant used is a quaternary nitrogen containing compound (“quat”) or indeed when a quat meraial is used in compositions or products in accordance with preferred embodiments of the invention, cationic activity may be used as a measure of the amount of quat actually used.

Cationic activity is appropriate for discussion in the context of quats. Cationic activity may be measured by several methods readily understood by those skilled in the art. One such method utilizes a standardized solution of an anionic material, such as sodium lauryl sulfate. This material is added to the solution containing the quat until full complexation of the quat's cations (the end point) has been reached. The end point can be measured potentiometrically or by the use of color indicators.

Typical tests involve titrating a sample of the quat, usually dissolved in a solvent, with the standardized solution of sodium lauryl sulfate until the endpoint is reached. As described in the co-pending and co-assigned U.S. patent application Ser. No. 09/438,631, incorporated by reference herein in its entirety, once the endpoint is reached, the cationic activity can be calculated according to the following formula:

$$\% cationic activity = \frac{mL \times N \times MW \times 100}{S . wt . \times 1000}$$

Where: mL=the number of mL of anionic material
N=the normality of the solution used
MW=the equivalent molecular weight of the quat being analyzed
S.wt.=the sample weight in grams.

For additional information regarding the methodology for measuring the cationic activity, see W. Schempp and H. T. Trau, Wochenblatt fur Papierfabrikation 19, 1981, pages 726-732, or J. P. Fischer and K. Lohr, Organic Coatings Science Technology, Volume 8, pages 227-249 (Marcel Dekker, Inc. 1986), both incorporated herein by reference in their entirety. While the use of quat raw materials having a high cationic activity is preferred (activity of at least about 35%, more preferably at least about 50%), use of lower cationic activities are also contemplated, particularly in finished products where the overall cationic activity may be less than 25%, less than 10% and even less than 5%.

Water

The preferred oil-in-water emulsion contains from about 25% to about 98%, preferably from about 65% to about 95%, more preferably from about 70% to about 90% water by weight of the topical carrier.

The hydrophobic phase is dispersed in the continuous aqueous phase. The hydrophobic phase may contain water insoluble or partially soluble materials such as are known in the art, including but not limited to the silicones described herein in reference to silicone-in-water emulsions, and other oils and lipids such as described above in reference to emulsions.

The topical compositions of the subject invention, including but not limited to lotions and creams, may contain a dermatologically acceptable emollient. Such compositions preferably contain from about 1% to about 50% of the emollient. As used herein, “emollient” refers to a material useful for the prevention or relief of dryness, as well as for the protection of the skin. A wide variety of suitable emollients are known and may be used herein. Sagarin, Cosmetics Science and Technology, 2^{nd} Edition, Vol. 1, pp. 32-43 (1972), incorporated herein by reference, contains numerous examples of materials suitable as an emollient. A preferred emollient is glycerin. Glycerin is preferably used in an amount of from or about 0.001 to or about 30%, more preferably from or about 0.01 to or about 20%, still more preferably from or about 0.1 to or about 10%, e.g., 5%.

Examples of suitable emollients include C₈₋₃₀ alkyl esters of C₈₋₃₀ carboxylic acids; C₁₋₆ diol monoesters and diesters of C₈₋₃₀ carboxylic acids; monoglycerides, diglycerides, and triglycerides of C₈₋₃₀ carboxylic acids, cholesterol esters of C₈₋₃₀ carboxylic acids, cholesterol, and hydrocarbons. Examples of these materials include diisopropyl adipate, isopropyl myristate, isopropyl palmitate, ethylhexyl palmitate, isodecyl neopentanoate, C₁₂₋₁₅ alcohols benzoates, diethylhexyl maleate, PPG-14 butyl ether, PPG-2 myristyl ether propionate, cetyl ricinoleate, cholesterol stearate, cholesterol isosterate, cholesterol acetate, jojoba oil, cocoa butter, shea butter, lanolin, lanolin esters, mineral oil, petrolatum, and straight and branched C₁₆-C₃₀ hydrocarbons.

Also useful are straight and branched chain fatty C₈-C₃₀ alcohols, for example, stearyl alcohol, isostearyl alcohol, ethenyl alcohol, cetyl alcohol, isocetyl alcohol, and mixtures thereof. Examples of other suitable emollients are disclosed in U.S. Pat. No. 4,919,934; which is incorporated herein by reference in its entirety.

Other suitable emollients are various alkoxylated ethers, diethers, esters, diesters, and trimesters. Examples of suitable alkoxylated ethers include PPG-10 butyl ether, PPG-11 butyl ether, PPG-12 butyl ether, PPG-13 butyl ether, PPG-14 butyl ether, PPG-15 butyl ether, PPG-16 butyl ether, PPG-17 butyl ether, PPG-18 butyl ether, PPG-19 butyl ether, PPG-20 butyl ether, PPG-22 butyl ether, PPG-24 butyl ether, PPG-30 butyl ether, PPG-11 stearyl ether, PPG-15 stearyl ether, PPG-10 oleyl ether, PPG-7 lauryl ether, PPG-30 isocetyl ether, PPG-10 glyceryl ether, PPG-15 glyceryl ether, PPG-10 butyleneglycol ether, PPG-15 butylene glycol ether, PPG-27 glyceryl ether, PPG-30 cetyl ether, PPG-28 cetyl ether, PPG-10 cetyl ether, PPG-10 hexylene glycol ether, PPG-15 hexylene glycol ether, PPG-10 1,2,6-hexanetriol ether, PPG-15 1,2,6-hexanetriol ether, and mixtures thereof.

Examples of alkoxylated diethers include PPG-10 1,4-butanediol diether, PPG-12 1,4-butanediol diether, PPG-14 1,4-butanediol diether, PPG-2 butanediol diether, PPG-10 1,6-hexanediol diether, PPG-12 1,6-hexanediol diether, PPG-14 hexanediol diether, PPG-20 hexanediol diether, and mixtures thereof. Preferred are those selected from the group consisting of PPG-10 1,4-butanediol diether, PPG-12 1,4-butanediol diether, PPG-10 1,6-hexandiol diether, and PPG-12 hexanediol diether, and mixtures thereof.

Examples of suitable alkoxylated diesters and trimesters are disclosed in U.S. Pat. Nos. 5,382,377, 5,455,025 and 5,597,555, assigned to Croda Inc., and incorporated herein by reference.

Suitable lipids include C₈-C₂₀ alcohol monosorbitan esters, C₈-C₂₀ alcohol sorbitan diesters, C₈-C₂₀ alcohol sorbitan Irimesters, C₈-C₂₀ alcohol sucrose monoesters, C₈-C₂₀ alcohol sucrose diesters, C₈-C₂₀ alcohol sucrose rimesters, and C₈-C₂₀ fatty alcohol esters of C₂-C₆₂ -hydroxy acids. Examples of specific suitable lipids are sorbitan diisostearate, sorbitan dioleate, sorbitan distearate, sorbitan isosotearate, sorbitan laurate, sorbitan oleate, sorbitan palmitate, sorbitan sesquioleate, sorbitan esquistearte, sorbitan stearate, sorbitan triiostearte, sorbitan trioleate, orbitan tristeate, sucrose cocoate, sucrodilaurate, sucrose distearate, sucrose laurate, sucrose myristate, sucrose oleate, sucrose palmitate, sucrose ricinoleate, sucrose stearate, sucrose tribehenate, sucrose tristearate, myristyl lactate, stearyl lactate, isostearyl lactate, cetyl lactate, palmityl lactate, cocoyl lactate, and mixtures thereof.

Other suitable emollients include mineral oil, petrolatum, cholesterol, dimethicone, dimethiconol, stearyl alcohol, cetyl alcohol, behenyl alcohol, diisopropyl adipate, isopropyl myristate, myristyl myristate, cetyl ricinoleate, sorbitan distearte, sorbitan dilaurate, sorbitan stearate, sorbitan laurate, sucrose laurate, sucrose dilaurate, sodium isostearyl lactylate, lauryl pidolate, sorbitan stearate, stearyl alcohol, cetyl alcohol, behenyl alcohol, PPG-14 butyl ether, PPG-15 stearyl ether, and mixtures thereof.

Lotions and creams according to the present invention generally contain a solution carrier system and one or more emollients. Lotions and creams typically contain from about 1% to about 50%, preferably from about 1% to about 20%, of emollient; from about 50% to about 90%, preferably from about 60% to about 80%, water; and the saccharose substitutes and the additional skin care active (or actives) in the above described amounts. Creams are generally thicker than lotions due to higher levels of emollients or higher levels of thickeners.

Ointments of the present invention may contain a simple carrier base of animal or vegetable oils or semi-solid hydrocarbons (oleaginous); absorption ointment bases which absorb water to form emulsions; or water soluble carriers, e.g., a water soluble solution carrier. Ointments may further contain a thickening agent, such as described in Sagarin, Cosmetics, Science and Technology, 2^{nd} Edition, Vol. 1, pp. 72-73 (1972), incorporated herein by reference, and/or an emollient. For example, an ointment may contain from about 2% to about 10% of an emollient; from about 0.1% to about 2% of a thickening agent; and the saccharose substitutes and the additional skin care active (or actives) in the above described amounts.

Compositions of this invention useful for cleansing (“cleansers”) are formulated with a suitable carrier, e.g., as described above, and preferably contain, in addition to the saccharose substitutes and the additional skin care active (or actives) in the above described amounts, from about 1% to about 90%, more preferably from about 5% to about 10%, of a dermatologically acceptable surfactant. The surfactant is suitably selected from anionic, nonionic, zwitterionic, amphoteric and ampholytic surfactants, as well as mixtures of these surfactants. Such surfactants are well known to those skilled in the detergency art. Nonlimiting examples of possible surfactants include isoceteth-20, sodium methyl cocoyl taurate, sodium methyl oleoyl taurate, and sodium lauryl sulfate. See U.S. Pat. No. 4,800,197, to Kowcz et al., issued Jan. 24, 1989, which is incorporated herein by reference in its entirety, for exemplary surfactants useful herein. Examples of a broad variety of additional surfactants useful herein are described in McCutcheon's Detergents and Emulsifiers, North American Edition (1986), published by Allured Publishing Corporation. The cleansing compositions can optionally contain, at their art-established levels, other materials which are conventionally used in cleansing compositions.

The physical form of the cleansing compositions is not critical. The compositions can be, for example, formulated as toilet bars, liquids, shampoos, bath gels, hair conditioners, hair tonics, pastes, or mousses. Rinse-off cleansing compositions, such as shampoos, require a delivery system adequate to deposit sufficient levels of actives on the skin and scalp. A preferred delivery system involves the use of insoluble complexes. For a more complete disclosure of such delivery systems, see U.S. Pat. No. 4,835,148, Barford et al., issued May 30, 1989.

The compositions of the invention may also include a hair setting agent to impart styling benefits upon application to hair. The hair setting polymers may be homopolymers, copolymers, terpolymers, etc. For convenience in describing the polymers hereof, monomeric units present in the polymers may be referred to as the monomers from which they can be derived. The monomers can be ionic (e.g., anionic, cationic, amphoteric, zwitterionic) or nonionic.

Examples of anionic monomers include unsaturated carboxylic acid monomers such as acrylic acid, methacrylic acid, maleic acid, maleic acid half ester, itaconic acid, fumeric acid, and crotonic acid; half esters of an unsaturated polybasic acid anhydride such as succinic anhydride, phthalic anhydride or the like with a hydroxyl group-containing acrylate and/or methacrylate such as hydroxyethyl acrylate and, hydroxyethyl methacrylate, hydroxypropyl acrylate and the like; monomers having a sulfonic acid group such as styrenesulfonic acid, sulfoethyl acrylate and methacrylate, and the like; and monomers having a phosphoric acid group such as acid phosphooxyethyl acrylate and methacrylate, 3-chloro-2-acid phosphooxypropyl acrylate and methacrylate, and the like.

Examples of cationic monomers include monomers derived from acrylic acid or methacrylic acid, and a quaternarized epihalohydrin product of a trialkylamine having 1 to 5 carbon atoms in the alkyl such as (meth)acryloxypropyltrimethylammonium chloride and (meth)acryloxypropyl-triethylammonium bromide; amine derivatives of methacrylic acid or amine derivatives of methacrylamide derived from methacrylic acid or methacrylamide and a dialkylalkanolamine having C₁-C₆ alkyl groups such as dimethylaminoethyl(meth)acrylate, diethylaminoethyl(meth)acrylate, dimethylaminopropyl(meth)acrylate, or dimethylaminopropyl(meth)acrylamide.

Examples of the amphoteric monomers include zwitterionized derivatives of the aforementioned amine derivatives of (meth)acrylic acids or the amine derivatives of (meth)acrylamide such as dimethylaminoethyl(meth)acrylate, dimethylaminopropyl(meth)acrylamide by a halogenated fatty acid salt such as potassium monochloroacetate, sodium monobromopropionate, aminomethylpropanol salt of monochloroacetic acid, triethanolamine salts of monochloroacetic acid and the like; and amine derivatives of (meth)acrylic acid or (meth)acrylamide, as discussed above, modified with propanesultone.

Examples of nonionic monomers are acrylic or methacrylic acid esters of C₁-C₂₄ alcohols, such as methanol, ethanol, 1-propanol, 2-propanol, 1-butanol, 2-methyl-1-propanol, 1-pentanol, 2-pentanol, 3-pentanol, 2-methyl-1-butanol, 1-methyl-1-butanol, 3-methyl-1-butanol, 1-methyl-1-pentanol, 2-methyl-1-pentanol, 3-methyl-1-pentanol, t-butanol, cyclohexanol, 2-ethyl-1-butanol, 3-heptanol, benzyl alcohol, 2-octanol, 6-methyl-1-heptanol, 2-ethyl-1-hexanol, 3,5-dimethyl-1-hexanol, 3,5,5-trimethyl-1-hexanol, 1-decanol, 1-dodecanol, 1-hexadecanol, 1-octadecanol, styrene; chlorostyrene; vinyl esters such as vinyl acetate; vinyl chloride; vinylidene chloride; acrylonitrile; alpha-methylstyrene; t-butylstyrene; butadiene; cyclohexadiene; ethylene; propylene; vinyl toluene; alkoxyalkyl(meth)acrylate, methoxy ethyl(meth)acrylate, butoxyethyl(meth)acrylate; allyl acrylate, allyl methacrylate, cyclohexyl acrylate and methacrylate, oleyl acrylate and methacrylate, benzyl acrylate and methacrylate, tetrahydrofurfuryl acrylate and methacrylate, ethylene glycol di-acrylate and -methacrylate, 1,3-butyleneglycol di-acrylate and -methacrylate, diacetonacrylamide, isobornyl (meth)acrylate, n-butyl methacrylate, isobutyl methacrylate, 2-ethylhexyl methacrylate, methyl methacrylate, t-butylacrylate, t-butylmethacrylate, and mixtures thereof.

Examples of anionic hair styling polymers are copolymers of vinyl acetate and crotonic acid, terpolymers of vinyl acetate, crotonic acid and a vinyl ester of an alpha-branched saturated aliphatic monocarboxylic acid such as vinyl neodecanoate; and copolymers of methyl vinyl ether and maleic anhydride, acrylic copolymers and terpolymers containing acrylic acid or methacrylic acid.

Examples of cationic hair styling polymers are copolymers of amino-functional acrylate monomers such as lower alkylamino alkyl acrylate or methacrylate monomers such as dimethyl aminoethylmethacrylate with compatible monomers such as N-vinylpyrrolidone or alkyl methacrylates such as methyl methacrylate and ethyl methacrylate and alkyl acrylates such as methyl acrylate and butyl acrylate.

The compositions of the invention may also include a wide range of miscellaneous ingredients. Some suitable miscellaneous ingredients commonly used in the cosmetic and personal care industry are described in The CTFA Cosmetic Ingredient Handbook, (10^{th} Ed., 2004), which is incorporated by reference herein. These ingredients will be used in amounts which are conventional.

Oleanolic acid, apigenin, and biotinyl-GHK, together, with or without other active substances and with or without at least one additional ingredient, may be used to impregnate any sort of textile, synthetic or natural fibers, wool or any materials liable to be used for the manufacture of clothing or underclothing, active materials of any sort, wipes, patches, compresses, cottons, and cotton buds, dressings, makeup-removing sponges, masks, or any other carrier liable to come into direct contact with the skin and scalp to enable continuous topical delivery. A more complete list of possible cosmetic compositions and other usually employed ingredients can be found in Robinson et al. U.S. Patent No. 6,492,326 B1, which issued on Dec. 10, 2002. The text from column 5, line 35 trough column 20, line 52 and column 20, line 61 through column 34, line 11 is hereby incorporated by reference. The text and claims referring to cosmetic compositions and ingredients as published in WO 03/028692, published on Apr. 10, 2003 and filed Oct. 1, 2002 as PCT/FR02/03344 are hereby incorporated by reference.

The present invention includes a method of treating hair loss comprising the steps of administering oleanolic acid, apigenin and biotinyl-GHK to the scalp of a subject in need of such treatment. To treat hair loss, the oleanolic acid, biotinyl-GHK, and apigenin should be administered at least once a day and preferably twice a day; once in the morning and once at night. This treatment should be continued indefinitely to continue to effectively treat hair loss. Preferably, these are administered at roughly the same time. In one embodiment, they are formulated in one or two total formulations and applied in these formulations.

Hair loss can be defined as having more hair fall out than is regrown over an entire hair cycle; through the anagen, catagen, and telogen phases, generally a period of between three and four years. Hair loss could also be measured over a one month period and would be defined as having more hair fall out than is regrown over that one month period. Preferably, hair loss could be measured over a longer period such as two or three months. A subject in need of a treatment for hair loss is an individual who has experienced or is experiencing hair loss or is at risk for experiencing hair loss in the future.

A successful treatment for hair loss can be measured in different ways: 1) reversing hair loss which causes less hair to fall out than is regrown over a period of time, 2) stopping hair loss which causes hair to fall out at approximately the same rate that it is regrown over a period of time, and 3) slowing hair loss which causes hair to fall out at a slower rate than it would had no treatment been administered. All three are contemplated by the method of treatment of the present invention.

Observations and measurements of hair loss can be made by counting the number of hairs on a portion of the scalp of an individual and at a later time counting the number of hairs on said portion of the scalp again and determining the difference between the two numbers. This counting can be done manually but it is preferably done by videotrichogram. If the amount of hairs present decreases over a period of one or more months, then hair loss is occurring. After treatment begins a further measurement can be taken. If this measurement shows a slowing of hair loss (where the amount of hair lost is smaller than before over the same period of time), a stopping of hair loss (where the amount of hair present stays the same), or a reversing of hair loss (where the amount of hair present increases) then the treatment can be considered successful. It is preferable that said measurements be taken at least a month apart, however, greater or lesser amounts of time between measurements are contemplated.

In a preferred embodiment, the oleanolic acid, biotinyl-GHK and apigenin are contained in a single formulation which is applied to the scalp, however, each ingredient could be packaged and applied separately or in combinations of, for example two of the three active ingredients in a first formulation and the third active ingredient in a second formulation. If the ingredients are packaged together they can be applied directly to the scalp or indirectly onto something else, such as a hand or a cloth, which is then used to wipe the formulation onto the scalp.

It is contemplated that the active ingredients could be applied separately, individually, or together. If the ingredients are applied separately it means that the ingredients are not all applied within the course of one hour. If the ingredients are administered individually it means that each ingredient is applied one at a time, or in a combination of two and one, either contemporaneously or within the scope of one hour. If the ingredients are applied together, then a single formulation containing all three ingredients is applied to the scalp. Said single formulation could be available containing the three ingredients or could be mixed together by an individual by mixing the three separate ingredients into a single formulation or mixing a combination of two ingredients with the third ingredient to create said single formulation.

The formulation/composition should be applied to the scalp of a subject in need of a treatment for hair loss at least once a day and more preferably twice a day, once in the morning and once at night. While application of the formulation/composition less frequently than once a day is contemplated, such a treatment is likely to be less effective than the preferred method of treatment. It is preferred that the treatment be continued indefinitely to continually treat hair loss, however, treatment for shorter periods of time is contemplated. It is also preferred for the formulation/composition to be left on the scalp after its administration.

The preferred formulation comprises about 0.03% by weight oleanolic acid, about 0.02% by weight biotinyl-GHK, about 0.05% by weight apigenin, and about 99.9% inactive ingredients. The inactive ingredients could include any of those ingredients discussed above and more specifically; butylenes glycol, water, PPG-26-buteth-26, PEG-40 hydrogenated castor oil. Preferably the inactive ingredients include about 87.4% of butylenes glycol, about 20% of water, about 2% of PPG-26-buteth-26 and about 1.5% of PEG-40 hydrogenated castor oil.

The preferred formulation/composition can be used to treat baldness by administering it to the scalp of a subject in need of a hair loss treatment. While more or less of the preferred formulation may be applied, between about 0.01 ml and 0.5 ml of the formulation per cm² of scalp should be applied. This should amount to between approximately 1 ml and 50 ml for the average subject. It is preferable that between about 5 ml and 10 ml be administered to the average subject which amounts to between about 0.05 and 0.1 ml/cm².

### EXAMPLES

The following nonlimiting examples are for the purpose of illustrating some of the uses and advantages of certain embodiments of the present invention.

### Example 1

### Study of Biotinyl-GHK on Cultured Hair Follicle Explants

A study was conducted on human skin explants cultured in PBS medium in a moist chamber at 21° C. Six explants containing hair follicles were incubated in the presence of 60 ppm biotinyl-GHK for 18 hours and compared to control explants exposed to a peptide-free excipient. This procedure was repeated in three batches. After the 18 hours an 8 mm biopsy was removed from the center of each well and immediately frozen in liquid nitrogen. 15 μm thick sections of the follicle were made using a freezing microtome which were then dried and fixed. Biotinyl-GHK in the sections was detected by immunolabeling coupled with streptavidine peroxidase. This was done to investigate for selective localization of the product around the pilial zone. The sections showed a clear peri-pilial localization of peptide biotinyl-GHK. This shows that biotinyl-GHK is a substantive peptide that exhibits specific localization around its target, the hair follicle.

### Example 2

### Anti-Aging Study on Cultured Hair Follicles

Excess hair follicles prepared in the context of a micrograft transplantation session were collected for culturing in a medium similar to that reported in, and herein encorporated by reference, Philpott et al., Whole Hair Follicule Culture, Dermatologic Clinics 595 (Oct. 14, 1996). Said hair follicles were then individually incubated at 37° C. under an air plus CO₂ (at 5%) atmosphere for 14 days. The explants were then divided into four groups: 1) a control group for the culture medium alone, 2) a positive control group which was exposed to a medium of 2 ppm Minoxidil®, 3) a test group exposed to the peptide biotinyl-GHK in a medium of 2 ppm biotinyl-GHK, and 4) a test group exposed to the peptide biotinyl-GHK in a medium of 5 ppm biotinyl-GHK. The culture medium was changed every 2 days. General morphology was observed on Day 1 and Day 14. Concomitantly, a fraction of the follicles were frozen for the purposes of conducting more advanced immunohistochemical studies. Growth in the follicles was monitored using a digital camera with images being taken on Day 0, Day 3, Day 5, Day 7, Day 11, and Day 14. The positive control group and the test group exposed to a medium of 2 ppm biotinyl-GHK demonstrated similar growth, both experiencing 58% more growth than was observed from the control group. The test group exposed to 5 ppm biotinyl-GHK experienced substantial growth; exhibiting 121% more growth than was observed from the control group.

### Example 3

### Anti-Aging Activity on the Root Sheath

The frozen microtome sections made on Day 0 and Day 14 from Example 2 were exposed to peroxidase-bound anti-Ki67 antibody. The dividing cells of these sections were stained dark brown. A count of cells showing the Ki67 marker was conducted on the lower section of the root sheath of the hair shaft under microscope. The count of the control bulb on Day 14 of the culture showed a decrease in mitotic keratinocytes, thereby reflecting cell aging. The positive control group, that exposed to a 2 ppm Minoxidil® medium, maintained proliferative activity as did the test groups exposed to 2 ppm and 5 ppm biotinyl-GHK mediums. However, the test group exposed to the 2 ppm biotinyl-GHK medium experienced superior proliferative activity to that obtained by Minoxidil® as reported by BOYERA et al., 1997. These results demonstrate that biotinyl-GHK exhibits an anti-aging effect on the keratinocytes of the control bulb.

### Example 4

### Stimulation of the Adhesion Proteins of the Root Sheath and Dermal Papilla

Samples of Example 2 were examined after 14 days of culturing. The control's dermoepidermal junction, on the outer sheath side, appeared flattened and showed essentially no basal lamina. In contrast, the hair follicles that were incubated in the 2 ppm and 5 ppm biotinyl-GHK medium exhibited clear basal laminas that maintained their sinusoidal character. These characteristics of the test sample are signs of a strongly adherent and living dermoepidermal junction.

### Example 5

### Detection of Laminin 5 and Collagen IV

Samples of Example 2, frozen at D0 and D14, were exposed to fluorescent antibodies specific to laminin 5 and collagen IV. The staining is observable as green fluorescence. Counterstaining of the nuclei was conducted using propidium iodide which resulted in red staining. Observations of the microtome sections under microscope were conducted on the inferior zone of the follicle above and below the bulb.

The control sample exhibited a loss in thickness of the laminin 5 band on the outer root sheath side after 14 days. The positive control sample, that exposed to a 2 ppm Minoxidil® medium, retained a thick and strip-like laminin 5 band after 14 days. The test sample exposed to a 2 ppm biotinyl-GHK medium retained a strong laminin 5 band both at the papilla level and in the outer root sheath after 14 days.

The control sample exhibited a drastic reduction in the collagen IV band in the root sheath after 14 days. Observations of the papilla were not taken for the control group as the papilla in this group lost its labeling during the 14 days. While not as substantial, the positive control sample, that exposed to a 2 ppm Minoxidil® medium, too experienced a loss of collagen IV density in the root sheath as well as the dermal papilla after 14 days. After 14 days, the test group exposed to the biotinyl-GHK medium retained a strong presence of collagen IV in the dermal papilla and was very thick and structured in the root sheath, such that the structure observed was almost identical to that of the control sample at D0.

### Example 6

### Gene Activation

A DNA array study employing a panel of 600 genes selected for their interest with respect to cell function was conducted on SkinEthic® reconstituted human epidermis samples incubated in the presence of a complex consisting of the 3 active substances of the present invention: biotinyl-GHK, oleanolic acid, and apigenin. The amount of the ingredients present in percent by weight was: biotinyl-GHK at 0.0006%, oleanolic acid at 0.0009%, and apigenin at 0.0015%. The samples were incubated for a period of 18 hours. The mRNA present in the cells was then reverse transcribed to yield DNA and amplified, by RT-PCR method, to obtain a legible signal against a set of control cultures. The results are disclosed below.

Genes up-regulated vs. the control (100%)
and coding for proteins

| Change in gene expression under exposure to | |
|---|---|
| test solution | % |
| Adhesion complex proteins | |
| Desmosomal proteins 1 & 3 (Desmogleins) | 135%/138% |
| Desmocollin 1 | 146% |
| Fibronectin receptor β-subunit | 134% |
| Vimentin | 138% |
| Laminin binding protein | 146% |
| Integrin β1 & β2 | 134%/144% |
| Antioxidant enzymes | |
| Thioredoxins peroxidases (TDPX2 & AO372) | 152%/174% |
| SOD (mitochondrial & cytosolic) | 150%/169% |
| Metallothioneins MTH & HMT | 188%/190% |
| CYP b-reductase | 160% |
| Stress proteins | |
| HSP 27 | 164% |
| HSP 90 | 139% |
| Anti-inflammatory proteins | |
| Interferon γ antagonist | 135% |
| Cell metabolism enzymes | |
| Mitochondrial trifunctional protein & Acyl CoA | 123%/128% |
| precursor | |
| Ornithine decarboxylase | 132% |
| Glutamine synthetase | 136% |
| Acetyl CoA transferase | 137% |
| Isocitrate dehydrogenase | 189% |
| iNOS | 143% |
| NADP isocitrate dehydrogenase | 189% |
| Proliferation/differentiation markers | |
| Proliferating cell nuclear antigen (PCNA) | 191% |
| Cytokeratins 10, 14 and 16 | 154%/150%/144% |
| Steroid receptor co-activator | 160% |

Genes down-regulated vs. the control (100%)
and coding for proteins

| Change in gene expression under exposure to | |
|---|---|
| test solution | % |
| Pro-inflammatory proteins | |
| Interferon γ receptor | −57% |
| Angiogenic and matrix remodeling factors | |
| Vitronectin | −52% |
| TIMP1/TIMP2 | −43%/−24% |
| Antichymotrypsin α1 | −43% |
| Lysyl hydroxylases 1&@ | −50%/−29% |
| Heparan sulfate proteoglycan | −40% |
| Collagen 1 subunit | −46% |
| Cell proliferation regulation | |
| Retinoic binding proteins CRABP1/CRABP2 | −34%/−63% |
| Vit. D3 receptor | −40% |

The up regulated genes reflect a cell profile oriented towards high growth activity with strongly expressed cell metabolism enzymes. Antioxidant protective enzymes were also associated with the up regulated genes since it is necessary to protect the cell against the oxygen free radicals systematically generated by the high level of metabolic activity. Markers of cell proliferation, such as proliferating cell nuclear antigen (PCNA), steroid receptor co-activator and cytokeratins 10, 14 and 16, were markedly up regulated. Associated protein HSP27 was also substantially up regulated, indicating pro-differentiation activity as demonstrated in Jonak, Subcorneal colocalization of the small heat shock protein, HSP27, with keratins and proteins of the cornified envelope, 147(1) Br J Dermatol. 13 (Jul. 24, 2002) which is herein incorporated by reference. The differentiation was accompanied by an increase in several adhesion proteins: those enabling cohesion between cells and the adhesion and the deployment of keratinocytes in cell layers (desmogleins, desmocollins); those involved in cell attachment to the basal lamina (laminin binding protein, vimentin, integrin α and β) and those adhesion proteins that ensure anchoring to the surrounding dermis (desmogleins, desmocollins). Specifically, the up regulated desmogleins are adhesion proteins that are indispensable for between-keratinocyte adhesion and which contribute to the formation of the outer root sheath of the hair and are also involved in anchoring the root sheath to dermal structures. Additionally, the up regulated vimetin is a constituent of the matrix synthesized by keratinocytes at the junction between the epithelial tissue and dermis which plays a role in the morphogenesis of hair. Further of note, cytokeratin 10, involved in differentiation, and cytokeratins 14 and 16, involved in morphogenesis of the hair and keratinocyte proliferation, were also up regulated.

Gene down regulation was reflected in the decreased expression of the interferon receptor which is associated with up regulation in the interferon antagonist; both of which demonstrate a strong anti-inflammatory contribution. The genes involved in matrix remodeling and angiogenesis were temporarily down regulated, while the cell proliferation pathways were intensified by a decrease in the factors with a negative impact on those pathways: CRABP ½ (cytoplasmic retinoic acid binding proteins) and vitamin D3 receptor (transcription factor for cell proliferation and differentiation).

### Example 7

### In Vivo Study

The subjects involved in this example consist of 35 male subjects of Caucasian origin, aged between 18 and 50 years and presenting more than 20% of their hair in the telogen phase. Individuals with grey hair on the vertex, diseases of the scalp, those taking corticosteroids, immunosuppressants or retinoids in the preceding six months, those taking anti-inflammatories in the preceeding week, those applying Minoxidil® or any local ‘anti-hair loss’ treatment, applied topically or taken orally, or trophic treatments of the hair in the preceding three months, those engaged in topical or oral treatment of the scalp (such as: anti-seborrheic, anti-dandruff daily friction) in the preceding four weeks, those who changed dietary or exercise habits during the study and/or those who immoderately used alcohol or tobacco were all excluded from the study.

17 of the subjects were randomly selected to receive a placebo and 18 of the subjects were randomly selected to receive a 3% dilute alcohol lotion composition containing oleanolic acid, apigenin, and biotinyl-GHK. The dilute solution comprised the ingredients and amounts as shown below:

| Starting material | INCI name | Supplier | % |
|---|---|---|---|
| Phase 1 | | | |
| Demineralized water | Water (Aqua) | | q.s. 100 |
| Citric acid | Citric Acid | | 0.26 |
| Sodium citrate | Sodium Citrate | | 1.20 |
| Incroquat CTC 30 | Cetrimonium Chloride | Croda | 1.00 |
| Phase 2 | | | |
| Ethanol | Ethanol | | 8.00 |
| Fragrance | Fragrance | | q.s. |
| Crillet 1 | Polysorbate 20 | Croda | 0.40 |
| Phase 3 | | | |
| PROCAPIL ™ | Butylene Glycol (and) | SEDERMA | 3% |
| | Water (Aqua) (and) PPG- | | |
| | 26-Buteth-26 (and) PEG-40 | | |
| | Hydrogenated Castor Oil | | |
| | (and) Apigenin (and) | | |
| | Oleanolic Acid (and) | | |
| | Biotinoyl Tripeptide-1 | | |

The PROCAPIL^{™}, composition of Phase 3, comprising oleanolic acid, apigenin, and biotinyl-GHK comprised the following ingredients and amounts:

| Ingredients | % | CAS Nr | EINECS Nr |
|---|---|---|---|
| Butylene Glycol | qs 100 | 107-88-0 | 203-529-7 |
| Water (Aqua) | ≈20 | 7732-18-5 | 231-791-2 |
| PPG-26-Buteth-26 | ≈2 | 9038-95-3 | n/a |
| PEG-40 | ≈1.5 | 61788-85-0 | n/a |
| Hydrogenated | | | |
| Castor Oil | | | |
| Apigenin | ≈0.05 | 520-36-5 | 208-292-3 |
| Oleanolic Acid | ≈0.03 | 508-02-1 | 208-081-6 |
| Biotinoyl-GHK | ≈0.02 | 299157-54-3 | n/a |

The placebo or test composition was applied to each subject's scalp twice daily using gentile massage. A physical examination of the scalp was conducted by a dermatologist at times T0 and T4 months. These examinations were conducted with a MORITEX SCOPEMAN® MS-500 videomicroscope fitted with a mobile 25× objective with optical fiber, connected to a digital image acquisition system. The images were analyzed by the COUNT-HAIR® program developed by Laboratories DERMSCAN. Image acquisition at T0 and after T4 months was conducted on the same shaved hair zone (about 1 cm²/200 hairs, on average), after marking. The parameters monitored were the length and growth rate of the hair and the proportion of hairs in the anagen phase and proportion in the telogen phase. At T0 and at the end of the study, T4 months, 24 hairs were sampled from the border of the alopecic zone using tweezers. Six subjects in the treatment group and 6 subjects in the placebo group underwent sampling. 12 hairs of each sample were fixed in Bouin's fluid and the other 12 hairs were frozen immediately prior to shipment for analysis.

Clinical studies to measure the impact of a treatment on hair scalp health use various criteria for evaluation. Those criteria selected for this study included: 1) Hair density (number of hair/cm²) to measure growth/regrowth of hair and 2) percentages of hair in anagen and telogen phases and ratios of these percentages to analyze the hair anchoring and hair vitality for hair still present on the scalp.

After four months of treatment with the test compound the subjects exhibited a marked improvement in the proportion of anagen phase hairs, significantly superior compared to T0. The placebo demonstrated no activity. In the test group 67% of the subjects exhibited an improvement in the anagen/telogen ratio and for 3 of the 12 improved subjects, the anagen/telogen ratio improved by 31.2%, 33.5% and 46.3% respectively. In contrast the placebo exhibited a trend towards a decrease in anagen hairs.

The mean growth rate for the placebo subjects exhibited no significant difference between the baseline at T0 and the end of the study at T4 months. In the placebo group the mean growth rate fell an average of 3% and the majority of subjects did not present any improvement (11 of 16 subjects). In the test group a trend toward an increase in the growth rate was shown by 8 subjects.

### Example 8

### Morphological Changes in the Hair

Hair samples of the subjects of Example 7 were taken (by pulling out) at the end of the study, T4 months and were observed under microscope. The results show that hair sampled from the test subjects exhibited a much more highly structured hair bulb for telogen hair than those exhibited by the subjects exposed to the placebo. Moreover, the samples of the test subjects exhibited root sheathes in anagen hairs that had thicker and more well defined cell bases. In the test group the root sheath was observed to be of high quality with a well structured basal lamina ensuring optimum dermal-epidermal adhesion on the outer side of the hair. The test group also showed anchoring zones with the hair shaft on the inner root sheath side. In contrast, the basal layer of the outer root sheath and the anchoring zones of the inner root sheath were not well structured in the control group.

### Example 9

### Immunofluorescence Readings of Collagen IV and Laminin 5

The hair samples of Example 8 were labeled with the fluorescent antibodies specific to laminin 5 and collagen IV as described in Example 5. At T4 months a greater concentration of laminin 5 was observed in the root sheath of the samples taken from the test subjects as compared with samples taken from the subjects exposed to placebo. Likewise, at T4 months, the concentration of collagen IV was greater in the hair bulb of the samples taken from the test subjects as compared with samples taken from the subjects exposed to placebo. This illustrates improvements in hair morphology by greater presence of adhesion complex proteins such as collagen IV and laminin 5.

### Formulation Examples

### Formulation Example 1

### Shampoo

| Ingredients | INCI Name | Supplier | % |
|---|---|---|---|
| Phase 1 | | | |
| Water deionised | Water (Aqua) | | qs 100 |
| Citric acid | | | 0.24 |
| Citrate | | | 1.20 |
| trisodique | | | |
| Preservative | | | qs |
| Phase 2 | | | |
| Sodium Laureth | Sodium Laureth Sulfate | Croda | 10.00 |
| Sulfate | | | |
| Crodasinic LS30 | Sodium Lauroyl | Croda | 10.00 |
| Incronam 30 | Sarcosinate | | 5.00 |
| Crothix Liquid | Cocamidopropyl Betaine | Croda | 5.00 |
| | PEG-150 Pentaerythrityl | | |
| | Tetrastearate (and) PEG- | | |
| | 6 Capryl/Capric | | |
| | Glycerides (and) Water | | |
| Phase 3 | | | |
| HAIRSPA ® | Glycerin, Lactitol, | SEDERMA | 1% |
| | Xylitol | | |
| Ground Citrus | | | 2% |
| fruits extract | | | |
| Apigenin | Apigenin | | 0.0020 |
| Oleanolic Acid | Oleanolic Acid | | 0.0012 |
| Biotinoyl-GHK | Biotinoyl Tripeptide-1 | | 0.0009 |
| Phase 4 | | | |
| Perfume | | | qs |

HAIRSPA ®: Glycerin, Lactitol, Xylitol is proposed by Sederma to reduce irritations, dryness, increased transepidermal water loss, itching and scaly appearance of the skin on the head (scalp).

### Formulation Example 2

### Conditioner

| Ingredients | INCI Name | Supplier | % |
|---|---|---|---|
| Phase 1 | | | |
| Water | Water (Aqua) | | qsp 100 |
| Sorbate de | | | 0.10 |
| potassium | | | |
| Phase 2 | | | |
| Crodazosoft DBQ | Quaternium 91 | Croda | 3.00 |
| | and | | |
| | Cetrimonium | | |
| | Methosulfate | | |
| | and Cetearyl | | |
| | Alcohol | | |
| Crodamol CS90 | Cetearyl | Croda | 4.00 |
| | Alcohol | | |
| Crillet 3 | Polysorbate | Croda | 1.00 |
| | 60 | | |
| Phase 3 | | | |
| Volpo G26 | Glycereth 26 | Croda | |
| Conservateur | | | |
| Phase 4 | | | |
| PROCAPIL ™ | Apigenin | SEDERMA | 3% |
| | (and) | | |
| | Oleanolic | | |
| | Acid (and) | | |
| | Biotinoyl | | |
| | Tripeptide-1 | | |
| Phase 5 | | | |
| NaOH 30% | | | 0.05 |
| Phase 6 | | | |
| Crillet 1 | Polysorbate | Croda | 1.00 |
| Parfum | 20 | | qs |

### Formulation Example 3

### Leave in Conditioner

| Ingredients | INCI Name | Supplier | H % |
|---|---|---|---|
| Phase 1 | | | |
| Water | Water (Aqua) | | qsp 100 |
| Potassium | | | 0.10 |
| Sorbate | | | |
| Acide Citrique | | | 0.22 |
| Citrate | Cetrimonium Chloride | | 1.20 |
| trisodique | | | |
| Incroquat CTC | | | 1.00 |
| 30 | | | |
| Phase 2 | | | |
| Procetyl AWS | PPG-5 Ceteth-20 | Croda | 2.00 |
| Preservative | | | |
| Phase 3 | | | |
| Matricaria | | | 2.00 |
| Chamomilla L | | | |
| ligules extract | | | |
| Olea europaea | | | 1.00 |
| leaf extract | | | |
| Biot-Gly-His- | Biotinoyl Tripeptide-1 | SEDERMA | 0.001 |
| Lys | | | |
| Phase 4 | | | |
| Crillet 1 | Polysorbate 20 | Croda | 1.00 |
| Perfume | | | qs |

### Formulation Example 4

### Anti-Hair Loss Tonic with PROCAPIL™ and Metod of Preparation

| | | % by wt |
|---|---|---|
| Part A | | |
| Citric Acid | | 0.26 |
| Trisodic Acid | | 1.20 |
| Potassium Sorbate | | 0.10 |
| Water deionised | | qsp 100 |
| Part B | | |
| Methyl Paraben | Croda | 0.20 |
| Procetyl AWS [PPG-5 Ceteth 20] | | 2.00 |
| Part C | | |
| PROCAPIL ™ | Sederma | 3.00 |
| Part D | | |
| Crillet 1 [Polysorbate 20] | Croda | 1.00 |
| Fragrance | | 0.10 |

Wherein PROCAPIL ™ has the formulation disclosed above in the Examples section.

Method

Weigh Part A. Melt Part B until dissolved. Add Part B to Part A with helix stirring (s=200). Add Part C to Part A+B with helix stirring (s=200). Mix part D and add it to Part A+B+C with helix stirring (s=200).

pH: 5.50

### Formulation Example 5

### Shampoo with CERAMIDE A2 and PROCAPIL™ and Method of Preparation

% by wt.

| | | |
|---|---|---|
| PART A | | |
| Methylchloroisothiazolinone (and) | | 0.20 |
| Methylisothiazolinone | | |
| Lactic Acid | | 0.05 |
| Water Deionised | | qs 100 |
| PART B | | |
| Sodium Laureth Sulfate | | 42.50 |
| Incronam 30 [Cocamidopropyl Betaine] | Croda | 10.00 |
| Crothix Liquid [PEG 150 Pentaerytrityl | Croda | 1.00 |
| Tetrastearate (and) | | |
| PEG 6 Caprylic/Capric | | |
| Glycerides (and) Water | | |
| Sodium Cocoamphoacetate | | 3.00 |
| Crodapearl NI Liquid [Sodium Laureth | Croda | 6.00 |
| Sulfate (and) | | |
| Hydroxyethyl Stearamide-MIPA | | |
| PART C | | |
| CERAMIDE A2 [Water (and) Glycerin | | 1.00 |
| (and) PEG 8 (and)Sederma | | |
| PEG-8/SMDI Copolymer (and) Palmitoyl | | |
| Myristyl Serinate (and) Sodium | | |
| Polyacrylate] | | |
| PROCAPIL [Butylene Glycol (and) Water | Sederma | 2.00 |
| (Aqua) (and) PPG-26-Buteth-26 | | |
| (and) PEG-40 Hydrogenated Castor Oil | | |
| (and) Apigenin (and) Oleanolic Acid | | |
| (and) Biotinoyl Tripeptide-1 | | |
| PART D | | |
| Perfume | | 0.10 |

Method

Weigh Part A. Add each ingredient of Part B, the one after the other, with round helix stirring. Homogenize carefully during 2 hours. Add Part C, then Part D. Homogenize.

pH=6.5-7.0

### Formulation Example 6

### Leave on Product and Method of Preparation

| | | % by wt |
|---|---|---|
| Part A | | |
| Crodazosoft DBQ [Quaternium 91 (and) | Croda | 3.00 |
| Cetrimonium Methosulfate (and) Cetearyl | | |
| Alcohol] | | |
| Crodacol CS 90 [Cetearyl Alcohol] | Croda | 1.00 |
| Crodamol SS [Cetyl Ester] | Croda | 3.00 |
| Part B | | |
| Potassium Sorbate | — | 0.10 |
| Water deionised | — | qsp 100 |
| Part C | | |
| Butylene Glycol | — | 5.00 |
| Methyl Parabens | — | 0.20 |
| Part D | | |
| DC 949 [Cyclohexasiloxane] | Dow Corning | 4.00 |
| Part E | | |
| Heliogenol ™ [Butylene Glycol (and) | Sederma | 0.20 |
| Helianthus Annuus (Sunflower) Seed | | |
| Extract] | | |
| Apigenin [Apigenin] | | 0.002 |
| Biotinoyl-Glycine-Histidine-Lysine | | 0.003 |
| [Biotinoyl Tripeptide-1] | | |
| Oleanolic acid [Oleanolic Acid] | | 0.005 |
| Part F | | |
| Fragrance | — | 0.10 |

Method

Weigh Part A and melt to 80° C. in water-bath. Mix Part C and melt it to 60° C. Weigh Part B. Add Part C to Part B and heat to 80° C. in water-bath. Pour Part A into Part B+C with staro stirring. Add Part D extemporaneously. Homogenize well. Allow to cool to around 35° C. Add Part F and Part E. Homogenize well.

pH: 6.50

### Formulation Example 7

### Hair Mask and Method of Preparation

| | | % by wt |
|---|---|---|
| Part A | | |
| Incroquat Behenyl TMC (Behentrimonium | Croda | 4.00 |
| Chloride and Cetearyl Alcohol) | | |
| Part B | | |
| Potassium Sorbate | — | 0.10 |
| Water deionised | — | qs 100 |
| Part C | | |
| Butylene Glycol | — | 5.00 |
| Methyl parabens | — | 0.20 |
| Part D | | |
| DC 345 (Cyclomethicone) | Dow Corning | 4.00 |
| Part E | | |
| Venuceane ™(Thermus Thermophilus | Sederma | 1.00 |
| Ferment and Glycerin) | | |
| Genistein | | 0.001 |
| Apigenin [Apigenin] | | 0.0008 |
| Biotinoyl-Glycine-Histidine-Lysine | | 0.0006 |
| [Biotinoyl Tripeptide-1] | | |
| Oleanolic acid [Oleanolic Acid] | | 0.0009 |
| Part F | | |
| Fragrance | — | 0.10 |

Method

Weigh Part A and melt to 85° C. in water-bath. Weigh Part C, dissolve at hot temperature and add to Part B. Melt Part B+C at 85° C. in water bath. Pour Part A into Part B+C with staro stirring. Homogenize carefully. Add Part D extemporaneously. Add Part E at around 35° C., then add Part F. Homogenize carefully and allow to cool.

pH =6.4

### Formulation Example 8

### Antiwrinkle Cream and Method of Preparation

| | | % by wt |
|---|---|---|
| Part A | | |
| Glycerine | — | 3.00 |
| Methyl Paraben | — | 0.20 |
| Part B | | |
| Water Deionised | — | Qsp 100 |
| Potassium Sorbate | — | 0.10 |
| Sodium Hydroxide | — | 0.90 |
| Part C | | |
| Crodamol AB [C-12-15 Alkyl Benzoate] | Croda | 7.00 |
| Crodafos MCA [Cetyl Phosphate] | Croda | 2.00 |
| Volpo S2 [Ceteareth 2] | Croda | 1.00 |
| Cithrol GMS AS [Glyceryl Stearate | Croda | 1.50 |
| (and) PEG 100 Steareate] | | |
| DC 345 [Cyclohexasiloxane] | Dow Corning | 2.00 |
| Dimethicone | — | 4.00 |
| Stearic Acid | — | 2.50 |
| Part D | | |
| Xanthan Gum | — | 0.60 |
| Part E | | |
| Moist 24 ™ [Imperata Cylindrica Root | Sederma | 3.00 |
| Extract (and) Water (and) Glycerin | | |
| (and) PEG 8 (and) Carbomer] | | |
| Part F | | |
| Matrixyl ™ [Glycerin (and) Water (and) | Sederma | 3.00 |
| Butylene Glycol (and) Carbomer (and) | | |
| Polysorbate 20 (and) Palmitoyl | | |
| Pentapeptide 3] | | |
| Part G | | |
| Biopeptide CL [Glyceryl | Sederma | 3.00 |
| Polymethacrylate (and) Propylene | | |
| Glycol (and) Palmitoyl Oligopeptide] | | |
| Part H | | |
| Darutoside [Butylene Glycol (and) | Sederma | 1.50 |
| Centella Asiatica Extract (and) | | |
| Darutoside] | | |
| Apigenin [Apigenin] | | 0;003 |
| Biotinoyl-GHK [Biotinoyl Tripeptide-1] | | 0.0005 |
| Oleanolic acid [Oleanolic Acid] | | 0.0007 |
| Part I | | |
| Fragrance | | 0.10 |

Method

Heat Part A to 70° C. until dissolution of the preservatives

Weigh and heat Part B to 60° C.

Sprinkle Part D in Part B with helix stirring (s=300 rpm) and allow to stir for 15 minutes

Pour Part A into Part (B+D), then heat to 75° C. in Bain-Marie

Heat Part C to 75° C. in Bain-Marie

Pour Part C into Part (A+B+D) when it has reached 75° C. (with helix stirring, s=300 rpm)

Add Part E at around 50° C. and homogenize well.

Add Part F, then Part G and H. Homogenize well.

Add Part I at around 35° C. and homogenize well

Adjust pH to 6.00-6.50

### Formulation Example 9

### Anti-Ageing Night Cream and Method of Preparation

| | | % by wt |
|---|---|---|
| Part A | | |
| Water deionised | — | Qsp 100 |
| Ultrez 10 (Carbomer) | Noveon | 0.10 |
| Part B | | |
| Glycerin | — | 5.00 |
| Part C | | |
| Dermaxyl ™ (C12-15 Alkyl Benzoate and | Sederma | 2.00 |
| Tribehenin and Ceramide 2 and PEG-10 | | |
| Rapeseed Sterol and Palmitoyl Oligopeptide) | | |
| Volpo S2 (Steareth 2) | Croda | 0.60 |
| Crodafos CES (Cetearyl Alcohol & Dicetyl | Croda | 4.00 |
| Phosphate and Ceteth 10 Phosphate) | | |
| Crodamol STS (PPG-3 Benzyl Ether Myristate) | Croda | 2.00 |
| Crodamol OSU (Dioctyl Succinate) | Croda | 7.00 |
| Crill 3 (Sorbitan Stearate) | Croda | 1.60 |
| Methyl Paraben | | 0.30 |
| Part D | | |
| Potassium Sorbate | — | 0.10 |
| Part E | | |
| Sodium Hydroxyde 30% | — | 0.35 |
| Water deionised | — | 3.50 |
| Part F | | |
| Apigenin [Apigenin] | | 0.005 |
| Biotinoyl-GHK [Biotinoyl Tripeptide-1] | | 1.10⁻⁶ |
| Oleanolic acid [Oleanolic Acid] | | 0.01 |
| Part G | | |
| Fragrance | | 0.10 |

Method

Sprinkle Ultrez 10 in the water. Allow to swell for 20 minutes. Add Part B to Part A. Heat Part A+B and Part C to 80° C. in Bain Marie. Mix well. Pour Part C into Part A+B with Staro stirring (s=30%). Homogenize well. Add Part D at around 70° C., then Part E at around 50° C. Add Part F and G at around 35° C.

pH: 5.8

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

Any range of numbers recited in the specification or paragraphs hereinafter describing various aspects of the invention, such as that representing a particular set of properties, units of measure, conditions, physical states or percentages, is intended to literally incorporate expressly herein by reference or otherwise, any number falling within such range, including any subset of numbers or ranges subsumed within any range so recited.

The term “about” when used as a modifier for, or in conjunction with, a variable, is intended to convey that the numbers and ranges disclosed herein are flexible and that practice of the present invention by those skilled in the art using concentrations, amounts, contents, properties such density, etc., that are outside of the range or different from a single value, will achieve the desired result, namely, providing a method of treating hair loss in a subject in need of such treatment comprising the steps of administering an effective amount of oleanolic acid to at least one first portion of the scalp of said subject; administering an effective amount of biotinyl-GHK to said at least one first portion of said scalp of said subject; and administering an effective amount of apigenin to said at least one first portion of said scalp of said subject; and providing a formulation for treating hair loss in a subject in need of such treatment comprising oleanolic acid, biotinyl-GHK, apigenin, and at least one additional ingredient.

Throughout the entire specification, including the claims, the word “comprise” and variations of the word, such as “comprising” and “comprises,” as well as “have,” “having,” “includes,” “include” and “including,” and variations thereof, means that the named steps, elements or materials to which it refers are essential, but other steps, elements or materials may be added and still form a construct with the scope of the claim or disclosure. When recited in describing the invention and in a claim, it means that the invention and what is claimed is considered to what follows and potentially more. These terms, particularly when applied to claims, are inclusive or open ended and do not exclude additional, unrecited elements or methods steps.

1. A method of treating hair loss in a subject in need of such treatment comprising the steps of: administering an effective amount of oleanolic acid to at least one first portion of the scalp of said subject; administering an effective amount of biotinyl-GHK to said at least one first portion of said scalp of said subject; and administering an effective amount of apigenin to said at least one first portion of said scalp of said subject.

2. The method of claim 1, wherein said oleanolic acid, apigenin, and biotinyl-GHK are administered together.

3. The method of claim 1, wherein said oleanolic acid, apigenin, and biotinyl-GHK are administered in a single formulation.

4. The method of claim 1, wherein said oleanolic acid, apigenin, and biotinyl-GHK are administered separately.

5. The method of claim 1, wherein said oleanolic acid, apigenin, and biotinyl-GHK are administered individually.

6. The method of claim 1, wherein said effective amount of oleanolic acid is at least about 0.0000000003 mg/cm³, said effective amount of biotinyl-GHK is at least about 0.0000000002 mg/cm³, and said effective amount of apigenin is at least about 0.0000000005 mg/cm³.

7. A formulation for treating hair loss in a subject in need of such treatment comprising oleanolic acid, biotinyl-GHK, apigenin, and at least one additional ingredient.

8. The formulation of claim 7, wherein said additional ingredient is selected from the group consisting of vitamins, panthenol (vitamin B5), tocopherol acetate, alcohol, peptides, scalp cleansers, stimulators/promoters of hair growth, improvers of keratin biosythesis, circulation activators, tonifying agents, fortifying agents, essential oils, soothing agents, surfactants, niacinamide, pyridoxine, DMDM hydantoin hydrolysed soy proteins, ornithine, chlorphenesin, arginine, organic silicium, chelating agents, glycols, antioxidants, carriers, stabilizers, dermopharmaceuticals, surfactants, conditioning agents, immoluents, solvents, coloring agents, and fragrances.

9. The formulation of claim 7, wherein said at least one additional ingredient comprises butylenes glycol, water, PPG-26-Buteth-26, and PEG-40 hydrogenated castor oil.

10. The formulation of claim 7, wherein said at least one additional ingredient is no less than 1% by weight of said formulation and the remainder of said formulation comprises oleanolic acid, apigenin, and biotinyl-GHK in any proportion so long as all are present in at least some amount.

11. The formulation of claim 10, wherein said at least one additional ingredient is between about 1% and about 99.99999% by weight of said formulation.

12. An additive for use in producing personal care, cosmetic and/or dermopharmaceutical compositions comprising oleanolic acid, biotinyl-GHK, apigenin, and at least one delivery agent.

13. The additive of claim 12, wherein the delivery agent is selected from the group consisting of solvents, dispersants, suspending agents, structuring agents, chelating agents, preservatives, stabilizers, pH adjusters and antimicrobial agents.

14. The additive of claim 12, wherein said oleanolic acid is no less than about 0.0003% by weight of said additive.

15. The additive of claim 12, wherein said biotinyl-GHK is no less than 0.0002% by weight of said additive.

16. The additive of claim 12, wherein said apigenin is no less than 0.0005% by weight of said additive.

17. The additive of claim 12, wherein said at least one delivery agent is no less than 1% by weight of said additive and the remainder of said additive comprises oleanolic acid, apigenin, and biotinyl-GHK in any proportion so long as all are present in at least some amount.

18. The additive of claim 12, wherein said at least one delivery agent is between about 1% and about 99.999% by weight of said additive.

19. The additive of claim 12, wherein said oleanolic acid is between about 0.0003% to about 1% by weight of said additive.

20. The additive of claim 19, wherein said oleanolic acid is present in an amount of about 0.03% by weight of said additive.

21. The additive of claim 12, wherein said biotinyl-GHK is between about 0.0002% to about 1% by weight of said additive.

22. The additive of claim 21, wherein said biotinyl-GHK is present in an amount of about 0.02% by weight of said formulation.

23. The additive of claim 12, wherein said apigenin is between about 0.0005% to about 1% by weight of said additive.

24. The additive of claim 23, wherein said apigenin is present in an amount of about 0.05% by weight of said additive.

25. A personal care, cosmetic, and/or dermopharmaceutical composition comprising oleanolic acid, biotinyl-GHK, apigenin, and at least one additional ingredient.

26. The personal care, cosmetic, and/or dermopharmaceutical composition of claim 25 wherein the at least one additional ingredient is selected from the group consisting of vitamins, panthenol (vitamin B5), tocopherol acetate, alcohol, peptides, scalp cleansers, promoters/stimulators of hair growth, improvers of keratin biosythesis, circulation activators, tonifying agents, fortifying agents, essential oils, soothing agents, surfactants, niacinamide, pyridoxine, DMDM hydantoin hydrolysed soy proteins, ornithine, chlorphenesin, arginine, organic silicium, chelating agents, glycols, and antioxidants.

27. The personal care, cosmetic, and/or dermopharmaceutical composition of claim 25 wherein the personal care, cosmetic, and/or dermopharmaceutical composition is in the form of a lotion, shampoo, conditioner, hair spray, gel, hair styling product, hair holding product, sunscreen, sunblock, soap, cream, emulsion, dispersion, solution, milk, suspension, cleanser, wash, scalp treatment lotion, or spray.

28. The personal care, cosmetic, and/or dermopharmaceutical composition of claim 25 wherein the oleanolic acid is present in an amount no less than about 0.0000003% by weight of the composition.

29. The personal care, cosmetic, and/or dermopharmaceutical composition of claim 28 wherein the oleanolic acid is present in an amount of between about 0.0000003% and 0.006% by weight of the composition.

30. The personal care, cosmetic, and/or dermopharmaceutical composition of claim 29 wherein the oleanolic acid is present in an amount of between about 0.00003% and 0.0015% by weight of the composition.

31. The personal care, cosmetic, and/or dermopharmaceutical composition of claim 25 wherein the apigenin is present in an amount no less than about 0.0000005% by weight of the composition.

32. The personal care, cosmetic, and/or dermopharmaceutical composition of claim 31 wherein the apigenin is present in an amount of between about 0.0000005% and 0.01% by weight of the composition.

33. The personal care, cosmetic, and/or dermopharmaceutical composition of claim 32 wherein the apigenin is present in an amount of between about 0.00005% and 0.0025% by weight of the composition.

34. The personal care, cosmetic, and/or dermopharmaceutical composition of claim 25 wherein the biotinyl-GHK is present in an amount no less than about 0.0000002% by weight of the composition.

35. The personal care, cosmetic, and/or dermopharmaceutical composition of claim 34 wherein the biotinyl-GHK is present in an amount of between about 0.0000002% and 0.004% by weight of the composition.

36. The personal care, cosmetic, and/or dermopharmaceutical composition of claim 35 wherein the biotinyl-GHK is present in an amount of between about 0.00002% and 0.001% by weight of the composition.
